(19) United States
(12) Patent Application Publication
(10) Pub. No.: US 2009/0104216 A1
(43) Pub. Date: Apr. 23, 2009

(54) Peptide-Based Influenza Vaccine Formulation

(75) Inventor: Jose Vidal Torres, Davis, CA (US)

Correspondence Address: EDWARDS ANGELL PALMER & DODGE LLP, P.O. BOX 55874, BOSTON, MA, 02205, US

(73) Assignee: Variation Biotechnologies, Inc., Ottawa (CA)

(21) Appl. No.: 11/921,436

(22) PCT Filed: Jun. 1, 2006
(86) PCT No.: PCT/CA2006/000891

§ 371 Date: Nov. 30, 2007

Related U.S. Application Data
(60) Provisional application No. 60/686,041, filed on Jun. 1, 2005.

(51) Int. Cl. A61K 39/00; A61K 38/16

(52) U.S. Cl. 424/186.1; 514/12

(57) ABSTRACT

Peptide-based anti-influenza formulations against influenza A and B are disclosed. The peptides are derived from influenza-based epitopes. The formulations are based on peptide mixtures which may be formulated so that variability is present at particular residues. The formulations can be used to prepare vaccines for preventing influenza in human, avian, murine or equine animals.

### CROSS REFERENCE TO RELATED APPLICATIONS

This application claims the benefit of priority of U.S. Provisional Patent Application No. 60/686,041, filed Jun. 1, 2005, which is incorporated herein by reference.

### FIELD OF THE INVENTION

The present invention relates generally to an anti-viral formulation, and in particular relates to a peptide-based influenza vaccine formulation.

### BACKGROUND OF THE INVENTION

Influenza is a common infectious disease of the respiratory system associated with the Orthomyxoviridae family of viruses. Because of the high degree of variability of the virus, vaccination is typically required on a yearly basis with a reformulated vaccine that takes into account strain variations. Despite the reformulation, it is not possible for a vaccine to include all the different strains actively infecting people in the world during a particular season.

While efficacious vaccines against influenza are currently available, they must be reformulated each year due to antigenic variation in the surface proteins of the virus. One roadblock to reformulation is the relatively long length of time required to formulate and prepare sufficient quantities of vaccine doses for responding to seasonal increases in flu infections. Typically, it can take over six months to prepare a vaccine; occasionally, a new or overlooked influenza strain becomes prominent during that six month period, leading to an epidemic.

Hemagglutinin (HA) is the major surface glycoprotein of influenza virus and a potent immunogen against which viral neutralizing antibodies are directed. Influenza viruses are typed as A or B on the basis of relatively stable intracellular nucleoproteins and envelope associated matrix proteins. Virus subtypes are based on two proteins in the viral envelope, HA and neuraminidase (NA), which undergo constant antigenic change. Fifteen distinct subtypes of HA and 9 subtypes of NA are recognized for influenza A viruses (De Jong, Rimmelzwaan, G. F., Fouchier, R. A. M., and Osterhaus, A. D. M. E. Journal of Infection, 2000; 40:218-228). The sudden appearance of a new subtype (antigenic shift) has caused three major pandemics in the past century: 1918 (Spanish Flu, H1N1), 1957 (Asian Flu, H2N2) and 1968 (Hong Kong Flu, H3N2). There has also been recent concern with avian flu, equine flu, and susceptibility in humans.

HA is the major envelope glycoprotein of influenza virus, and mediates the penetration of virus into host cells (Wiley, D. C., et al., Nature 1981; 289(29):373-377; Wilson, I A, et al., Nature 1981; 289, 366-373; Caton, et al., Cell 1982:417-427). The native HA is formed by the association of three HA monomers which, as a precondition of virus infectivity, are cleaved enzymatically into the amino-terminal HA1 and carboxy-terminal HA2. Based on the three dimensional structure of HA1, antigenic sites have been mapped by determining the amino acid changes of antigenic variants (Wiley, supra). The antigenic variations were mostly seen surrounding the receptor binding region of HA, including residues around the antibody inaccessible receptor binding pockets. Thus, there has been a desire to elucidate peptide-based approaches as immunological therapies. However, a major concern of peptide-based subunit vaccines is the ability of linear peptides to induce antibodies that can recognize conformational B cell and T cell epitopes. Monoclonal antibodies to these antigenic sites neutralize influenza virus infectivity when the exact sequences are present. Both T and B cell epitopes are found on these sites (Atassi et al., Advances in Experimental Medicine and Biology, 1989; 251:49-635-6; Torres et al, Immunology Letters, 1988; 19(1):49-53).

Multiplication of influenza virus in the presence of monoclonal antibody directed against one of the antigenic sites on HA results in the selection of escape variants, which were a very small minority in the parent virus (Webster et al., Virology 1983; 126(2):587-99; Webster et al., Virology 1980; 104(1):139-48; Yewdell et al., Nature 1979; 279(5710):246-8). Consistent with these in vitro data, individuals immunized against one strain of virus and infected with another produce a wide array of antigenic variants. These data suggest that population immunity against those epitopes are a driving force for the selection of new strains of influenza viruses.

The vaccine formulation developed each year in the US is determined by the Department of Food and Drug Administration Vaccines and the Related Biologicals Advisory Committee. The World Health Organization (WHO) similarly operates a global surveillance network of laboratories, for detection of new influenza variants (Lavanchy, Vaccine 1999; 17:S24-S25). Selection is based on antigenic analysis of recently isolated influenza viruses, the patterns of spread of antigenic variants, and the antibody responses of recently vaccinated individuals. However, because of the delays in vaccine formulation outlined above, it has been difficult to effectively and efficiently produce sufficient quantities of vaccine to meet the changing requirements in immunization strategies. It is, therefore, desirable to efficiently provide a more efficacious influenza vaccine formulation.

### SUMMARY OF THE INVENTION

It is an object of the present invention to obviate or mitigate at least one disadvantage of previous influenza vaccine formulations.

The present invention provides peptide-based influenza vaccine formulations derived from epitopes from influenza HA. Advantageously, the vaccine formulations of the present invention improve the humoral response in animal models when compared with commercial vaccines. Because of the peptide variants in the formulations, the present invention can provide broad protection against different influenza virus strains.

Surprisingly, it was found that peptide-based influenza vaccine formulations in accordance with the present invention, which represent the antigenic diversity of influenza virus in protective HA epitopes, elicit protective immunity that is more broadly reactive than that induced with a commercial vaccine that is based on only a few isolates of influenza.

In a first aspect, the present invention provides a peptide-based anti-influenza formulation comprising at least one peptide selected from the group consisting of SEQ ID NOs: 1 to 248.

In one embodiment of the present invention there is provided a formulation comprising at least four peptide sequences selected from the group consisting of SEQ ID NOs: 1 to 248. In another embodiment, the formulation can comprise at least two peptides selected from the group consisting of SEQ ID NOs: 1 to 64 and at least two peptides selected from the group consisting of SEQ ID NOs: 133 to 180. The formulation can also comprise SEQ ID NOs: 1 to 64 and SEQ ID NOs: 133 to 180.

The present invention also provides a formulation comprising at least one peptide sequence selected from the group consisting of SEQ ID NOs: 185 to 248. In one embodiment, the formulation can comprise at least two peptide sequences from each of at least two of the following groups: SEQ ID NOs: 185 to 200; SEQ ID NOs: 201 to 216; SEQ ID NOs: 217 to 232; or SEQ ID NOs: 233 to 248.

The present invention also provides a formulation comprising at least one peptide sequence selected from the group consisting of SEQ ID NOs: 65 to 128. In one embodiment, the formulation can comprise at least two peptide sequences from each of at least two of the following groups: SEQ ID NOs: 65 to 80; SEQ ID NOs: 81 to 96; SEQ ID NOs: 97 to 112; or SEQ ID NOs: 113 to 128.

The formulations of the present invention can be used to prepare vaccines. An adjuvant, such as alum, or other substituent may be used in the preparation of the vaccine. The vaccine can be used in the treatment of influenza in animals such as humans, mice, horses or birds.

The formulation of the present invention is broadly reactive against influenza A and B. Advantageously, the formulation of the present invention can be prepared synthetically in as little as 6 weeks.

Other aspects and features of the present invention will become apparent to those ordinarily skilled in the art upon review of the following description of specific embodiments of the invention in conjunction with the accompanying figures.

### BRIEF DESCRIPTION OF THE DRAWINGS

Embodiments of the present invention will now be described, by way of example only, with reference to the attached Figures, wherein:

FIG. 1 shows HPLC analysis of equine influenza discosite constructs (INFE-HA-1-V1 to V4; A to D) of the present invention.

FIG. 2 shows HPLC analysis of human influenza A discosite constructs (INF-HA-1-V1 to V4; A to D) of the present invention.

FIG. 3 shows HPLC analysis of human and avian influenza discosite constructs (INF-HA-2-V1 to V4; A to D) of the present invention.

FIG. 4 shows HPLC analysis of influenza B discosite constructs (INF-HB-1-V1 to V4; A to D) of the present invention.

FIGS. 5A to 5D illustrate mass spectrometry data for equine influenza discosite constructs (INFE-HA-1-V1 to V4; FIGS. 5A to 5D) of the present invention.

FIGS. 6A to 6D illustrate mass spectrometry data for human influenza A discosite constructs (INF-HA-1-V1 to V4; FIGS. 6A to 6D) of the present invention.

FIGS. 7A to 7D illustrate mass spectrometry data for human and avian influenza discosite constructs (INF-HA-2-V1; FIGS. 7A to 7D) of the present invention.

FIGS. 8A to 8D illustrate mass spectrometry data for human influenza B discosite constructs (INF-HB-1-V1 to V4; FIGS. 8A to 8D) of the present invention.

FIG. 9 illustrates induction of humoral immunity by a vaccine of the present invention after immunization.

FIG. 10 shows a survival plot of vaccinated mice against challenge with H3N2.

FIG. 11 shows percent weight loss in challenged mice vaccinated with a vaccine of the present invention INF-01P (INF-HA-1-V1-V4).

FIG. 12 shows induction of humoral immunity by INFE-01P (INFE-HA-1-V1-V4) vaccination in mice as measured by HAI titres.

FIG. 13 illustrates the results of a hemagluttination assay performed in murine vaccine study.

FIG. 14 shows results of the influenza vaccine ELISA test based on data presented in Table 22.

### DETAILED DESCRIPTION

Generally, the present invention provides an anti-viral formulation, and more specifically, a peptide-based anti-influenza formulation comprising at least one peptide selected from the group consisting of SEQ ID NOs: 1 to 248.

The formulation of the present invention is a cocktail comprising one or more peptides. The formulation can comprise at least four peptide sequences selected from the group consisting of SEQ ID NOs: 1 to 248. In one example, the formulation can comprise at least two peptides selected from the group consisting of SEQ ID NOs: 1 to 64 and at least two peptides selected from the group consisting of SEQ ID NOs: 133 to 180. A specific example comprises SEQ ID NOs: 1 to 64 and SEQ ID NOs: 133 to 180. In another specific example, the formulation comprises SEQ ID NOs: 1 to 64. In yet another specific example, the formulation comprises SEQ ID NOs: 185 to 248.

In another example, the formulation can comprise 2ⁿ peptide sequences from each of at least two of the following groups: a) SEQ ID NOs: 1 to 16; b) SEQ ID NOs: 17 to 32; c) SEQ ID NOs: 33 to 48: or d) SEQ ID NOs: 49 to 64, wherein n is 1 to 4. Further, the formulation can comprise from at least two of groups a) to d): a) 2^{m} peptide sequences from SEQ ID NOs: 133 to 140; b) 2ⁿ peptide sequences from SEQ ID NOs: 141 to 156; c) 2ⁿ peptide sequences from SEQ ID NOs: 157 to 172; or d) 2^{m} peptide sequences from SEQ ID NOs: 173 to 180, wherein m is 1 to 3 and n is 1 to 4. This formulation can be used in the preparation of a human anti-influenza vaccine.

The human anti-influenza formulations described herein can be used alone or in combination.

In another example, the formulation of the present invention can comprise at least one peptide sequence selected from the group consisting of SEQ ID NOs: 185 to 248. As one example, the formulation can comprise 2ⁿ peptide sequences from each of at least two of the following groups: a) SEQ ID NOs: 185 to 200; b) SEQ ID NOs: 201 to 216; c) SEQ ID NOs: 217 to 232; or d) SEQ ID NOs: 233 to 248, wherein n is from 1 to 4. This formulation can be used in the preparation of an equine anti-influenza vaccine.

In another example, the formulation of the present invention can comprise at least one peptide sequence selected from the group consisting of SEQ ID NOs: 65 to 128. As one example, the formulation can comprise 2ⁿ peptide sequences from each of at least two of the following groups: a) SEQ ID NOs: 65 to 80; b) SEQ ID NOs: 81 to 96; c) SEQ ID NOs: 97 to 112; or d) SEQ ID NOs: 113 to 128, wherein n is from 1 to 4. This formulation can be used in the preparation of an avian anti-influenza vaccine.

In an example of a formulation where there is no variability represented by the peptide sequences, the formulation can comprise at least one of SEQ ID NOs: 129 to 132 or SEQ ID NOs: 181 to 184. As one specific example, the formulation can comprise SEQ ID NOs: 129 to 132. In another specific example, the formulation can comprise SEQ ID NOs: 181 to 184.

The present invention also provides a vaccine comprising a formulation including at least one peptide selected from the group consisting of SEQ ID NOs: 1 to 248, together with a pharmaceutically-acceptable diluent or carrier. The vaccine can further comprise an adjuvant which can be, for example, alum.

The formulation can be used for the preparation of a vaccine for preventing or treating influenza in an animal in need thereof. The animal can be human, murine, equine or avian.

The invention relates to the use of a formulation comprising at least two peptides selected from the group consisting of SEQ ID NOs: 1 to 64 and at least two peptides selected from the group consisting of SEQ ID NOs: 133 to 180 for the preparation of a vaccine for treating human influenza.

The invention also relates to the use of a formulation comprising at least one peptide sequence selected from the group consisting of SEQ ID NOs: 185 to 248 for the preparation of a vaccine for treating equine influenza.

The invention further relates to the use of the formulation comprising at least one peptide sequence selected from the group consisting of SEQ ID NOs: 65 to 128 for the preparation of a vaccine for treating avian influenza.

The vaccines prepared in accordance with the present invention can be used for preventing or treating influenza.

The sequences of these peptides are determined based on analysis of the crystal structure of influenza hemagglutinin (HA) protein to determine peptide epitopes. Hemagglutinin is the major surface glycoprotein of influenza virus and a potent immunogen against which viral neutralizing antibodies are directed. The linear peptide epitopes in the cocktail mimic discontinuous epitopes on the HA protein surface. Using bioinformatics software that analyzes the antigenic variation of HA proteins from thousands of human influenza isolates, degenerative peptide cocktails based on these epitopes can be prepared which represent the antigenic variation of HA within these epitopes. Thus, the influenza vaccine formulations of the present invention comprise a cocktail of peptides that represent major epitopes of the HA protein.

The vaccine may be formulated with or without representing variation at specific residues for each peptide. When variation is not represented, the peptide formed may be referred to herein as a Discotope™ construct. A discotope construct is a linear sequence synthetic construct that approximates the position of primary sequence sections that compose discontinuous epitopes. The individual sections are constructed in sequence to elicit immune responses that recognize the discontinuous epitopes found in the original intact protein.

Discontinuous epitopes are composed of two or more segments of the primary sequence of a protein that when properly folded come together and are bound by specific antibodies. They are not recognized by antibodies when the secondary structure is lost and therefore have not been represented by a continuous linear peptide.

When variation is present at particular residues that are known to have different amino acids represented according to different sequences for that particular pathogen, the formulation comprises a number of peptides, which may be collectively referred to herein as a Discosite™ construct.

In order to formulate a mixture of peptides, it is possible to use the method of Torres as outlined in U.S. patent application Ser. No. 10/072,084, which is herein incorporated by reference.

### Design of Discotope/Discosite Constructs

In the vaccine formulations of the present invention, influenza hemagglutinin is used to design linear sequences that represent four discontinuous epitopes. Four peptides (discotope constructs) that mimic discontinuous B and T cell epitopes on four antigenic sites of HA were designed. Each discotope construct is synthesized using solid phase peptide synthesis. Sequences of more than 200 human isolates of Influenza A were obtained from Genbank™ and Swiss Protein™ databases and aligned to study the composition of these epitopes.

An influenza vaccine formulation can comprise one or more discotope constructs of SEQ ID NOs: 129 to 132, and/or SEQ ID NOs: 181 to 184.

### Design of Vaccine Formulations

In the context of the present invention, a vaccine formulation is a cocktail of peptides that are used in the preparation of an influenza vaccine. The vaccine can comprise the cocktail of peptides and other substituents known in the art that would be found acceptable for inclusion. These substituents can include, but are not limited to, adjuvants, diluents and/or carriers.

The vaccine formulations of the present invention are particularly suitable for preparing vaccines in the treatment of human, equine and/or avian influenza. However, it will be appreciated that any combination of peptide sequences, or formulations comprising these peptide sequences, may be used in other influenza phenotypes.

Peptide vaccines can be prepared with a pool of one or more peptide sequences representing epitopes contained in the three-dimensional structure of HA (SEQ ID NOs: 1-248). The vaccines comprise one or more discotope constructs (peptides containing non-variable amino acid residues) or one or more discosite constructs (peptides containing variable amino acid residues). A discosite construct of the present invention is derived from one of these epitopes. Thus, a discosite construct formulation comprises one or more peptide sequences derived from the epitope containing the variable residues.

Each discosite construct of the present invention represents 2^{x} possible peptide sequences based on x varied residues. For example, a discosite construct having 3 or 4 variable residues represents 2³=8 or 2⁴=16 sequences, respectively. Therefore, in the context of the present invention, a discosite construct as referred to herein includes the epitope sequence containing the variable residues and the one or more possible sequences derived therefrom.

The vaccines of the present invention can comprise at least two peptide sequences from a given discosite construct, derived from at least two epitopes contained in the vaccine, for a total of at least 4 peptide sequences (from SEQ ID NOs: 1 to 248) in the vaccine. In some embodiments of the present invention, the human influenza vaccine formulation can comprise at least 4 human influenza-A (INF-HA-1-V1-V4) and/or 4 human influenza-B HA discosite construct sequences (INF-HB-1-V1-V4). This can include at least two peptides from SEQ ID NOs: 1 to 64 and/or at least two peptides from SEQ ID NOs: 133 to 180.

Alternatively, this can include at least two peptide sequences from each of at least two of the following groups: SEQ ID NOs: 1 to 16; SEQ ID NOs: 17 to 32; SEQ ID NOs: 33 to 48: or SEQ ID NOs: 49 to 64; and/or at least two peptide sequences from each of at least two of the following groups: SEQ ID NOs: 133 to 140; SEQ ID NOs: 141 to 156; SEQ ID NOs: 157 to 172: and/or SEQ ID NOs: 173 to 180.

Embodiments of the equine influenza vaccine can comprise at least one peptide sequence derived from the 4 equine discosite constructs (INFE-HA-1-V1-V4; SEQ ID NOs: 185 to 248), In some embodiments, the equine vaccine formulation can comprise at least two peptide sequences from each of at least two of the following groups: SEQ ID NOs: 185 to 200; SEQ ID NOs: 201 to 216; SEQ ID NOs: 217 to 232; and/or SEQ ID NOs: 233 to 248.

Embodiments of the avian influenza vaccine can comprise one or more peptide sequences of the 4 avian discosite constructs (INF-HA-2-V1-V4; SEQ ID NOs; 65 to 128). In some embodiments, the avian vaccine formulation can comprise at least two peptide sequences from each of at least two of the following groups: SEQ ID NOs: 65 to 80; SEQ ID NOs: 81 to 96; SEQ ID NOs: 97 to 112; and/or SEQ ID NOs: 113 to 128.

It will be appreciated by the person of ordinary skill in the art that additional sequences may or may not be added as required. The peptide sequences for use in the vaccine formulations of the present invention are grouped according to the discosite (Tables 1 to 16) or discotope (Tables 17 to 18) construct.

Tables 1 to 4 list discosite constructs of influenza A (human HA-1) epitope sequence.

Tables 5 to 8 list discosite constructs of influenza A (avian HA-2) epitope sequences.

Tables 9 to 12 list discosite constructs of influenza B (human HB-1) epitope sequences.

Tables 13 to 16 list discosite constructs of equine influenza (equine HA-1) epitope sequences.

In each of the discosite constructs listed in the Tables, the variable residue(s) is/are shown below the corresponding residue in the construct.

TABLE 1

Discosite constructs for INF-HA-1-V1
YACKRGGKSSGSSYPVLNVSY (SEQ ID NO: 1)
----H------------S-TM

| | | |
|---|---|---|
| Peptide Name | Sequence | MW |

| | | | |
|---|---|---|---|
| INF-HA-1-V1/1 | YACKRGGKSSGSSYPVLNVSY | (SEQ ID NO: 1) | 2223.47 |
| INF-HA-1-V1/2 | YACKRGGKSSGSSYPVLNVSM | (SEQ ID NO: 2) | 2191.49 |
| INF-HA-1-V1/3 | YACKRGGKSSGSSYPVLNVTY | (SEQ ID NO: 3) | 2237.5 |
| INF-HA-1-V1/4 | YACKRGGKSSGSSYPVLNVTM | (SEQ ID NO: 4) | 2205.52 |
| INF-HA-1-V1/5 | YACKRGGKSSGSSYPVLSVSY | (SEQ ID NO: 5) | 2196.44 |
| INF-HA-1-V1/6 | YACKRGGKSSGSSYPVLSVSM | (SEQ ID NO: 6) | 2164.47 |
| INF-HA-1-V1/7 | YACKRGGKSSGSSYPVLSVTY | (SEQ ID NO: 7) | 2210.47 |
| INF-HA-1-V1/8 | YACKRGGKSSGSSYPVLSVTM | (SEQ ID NO: 8) | 2178.49 |
| INF-HA-1-V1/9 | YACKHGGKSSGSSYPVLNVSY | (SEQ ID NO: 9) | 2204.42 |
| INF-HA-1-V1/10 | YACKHGGKSSGSSYPVLNVSM | (SEQ ID NO: 10) | 2172.45 |
| INF-HA-1-V1/11 | YACKHGGKSSGSSYPVLNVTY | (SEQ ID NO: 11) | 2218.45 |
| INF-HA-1-V1/12 | YACKHGGKSSGSSYPVLNVTM | (SEQ ID NO: 12) | 2186.47 |
| INF-HA-1-V1/13 | YACKHGGKSSGSSYPVLSVSY | (SEQ ID NO: 13) | 2177.4 |
| INF-HA-1-V1/14 | YACKHGGKSSGSSYPVLSVSM | (SEQ ID NO: 14) | 2145.42 |
| INF-HA-1-V1/15 | YACKHGGKSSGSSYPVLSVTY | (SEQ ID NO: 15) | 2191.42 |
| INF-HA-1-V1/16 | YACKHGGKSSGSSYPVLSVTM | (SEQ ID NO: 16) | 2159.45 |

TABLE 2

Discosite constructs for INF-HA-1-V2
KKGSVHHPSTITEQTSLYVNA (SEQ ID NO: 17)
-S-------------T--QQ-

| | | |
|---|---|---|
| Peptide Name | Sequence | MW |

| | | | |
|---|---|---|---|
| INF-HA-1-V2/1 | KKGSVHHPSTITEQTSLYVNA | (SEQ ID NO: 17) | 2297.53 |
| INF-HA-1-V2/2 | KKGSVHHPSTITEQTSLYVQA | (SEQ ID NO: 18) | 2311.55 |
| INF-HA-1-V2/3 | KKGSVHHPSTITEQTSLYQNA | (SEQ ID NO: 19) | 2326.53 |
| INF-HA-1-V2/4 | KKGSVHHPSTITEQTSLYQQA | (SEQ ID NO: 20) | 2340.55 |
| INF-HA-1-V2/5 | KKGSVHHPSTITEQTTLYVNA | (SEQ ID NO: 21) | 2311.55 |
| INF-HA-1-V2/6 | KKGSVHHPSTITEQTTLYVQA | (SEQ ID NO: 22) | 2325.58 |
| INF-HA-1-V2/7 | KKGSVHHPSTITEQTTLYQNA | (SEQ ID NO: 23) | 2340.55 |
| INF-HA-1-V2/8 | KKGSVHHPSTITEQTTLYQQA | (SEQ ID NO: 24) | 2354.58 |
| INF-HA-1-V2/9 | KSGSVHHPSTITEQTSLYVNA | (SEQ ID NO: 25) | 2256.43 |
| INF-HA-1-V2/10 | KSGSVHHPSTITEQTSLYVQA | (SEQ ID NO: 26) | 2270.46 |
| INF-HA-1-V2/11 | KSGSVHHPSTITEQTSLYQNA | (SEQ ID NO: 27) | 2285.43 |
| INF-HA-1-V2/12 | KSGSVHHPSTITEQTSLYQQA | (SEQ ID NO: 28) | 2299.46 |
| INF-HA-1-V2/13 | KSGSVHHPSTITEQTTLYVNA | (SEQ ID NO: 29) | 2270.46 |
| INF-HA-1-V2/14 | KSGSVHHPSTITEQTTLYVQA | (SEQ ID NO: 30) | 2284.48 |
| INF-HA-1-V2/15 | KSGSVHHPSTITEQTTLYQNA | (SEQ ID NO: 31) | 2299.46 |
| INF-HA-1-V2/16 | KSGSVHHPSTITEQTTLYQQA | (SEQ ID NO: 32) | 2313.48 |

TABLE 3

Discosite constructs for INF-HA-1-V3
DVLFSVESPNNKNKDPIDTCD (SEQ ID NO: 33)
------K-V-----ES-----

| | | |
|---|---|---|
| Peptide Name | Sequence | MW |

| | | | |
|---|---|---|---|
| INF-HA-1-V3/1 | DVLFSVESPNNKNKDPIDTCD | (SEQ ID NO: 33) | 2350.52 |
| INF-HA-1-V3/2 | DVLFSVESPNNKNKDSIDTCD | (SEQ ID NO: 34) | 2340.48 |
| INF-HA-1-V3/3 | DVLFSVESPNNKNKEPIDTCD | (SEQ ID NO: 35) | 2364.55 |
| INF-HA-1-V3/4 | DVLFSVESPNNKNKESIDTCD | (SEQ ID NO: 36) | 2354.51 |
| INF-HA-1-V3/5 | DVLFSVESVNNKNKDPIDTCD | (SEQ ID NO: 37) | 2352.54 |
| INF-HA-1-V3/6 | DVLFSVESVNNKNKDSIDTCD | (SEQ ID NO: 38) | 2342.5 |
| INF-HA-1-V3/7 | DVLFSVESVNNKNKEPIDTCD | (SEQ ID NO: 39) | 2366.56 |
| INF-HA-1-V3/8 | DVLFSVESVNNKNKESIDTCD | (SEQ ID NO: 40) | 2356.52 |
| INF-HA-1-V3/9 | DVLFSVKSPNNKNKDPIDTCD | (SEQ ID NO: 41) | 2349.58 |
| INF-HA-1-V3/10 | DVLFSVKSPNNKNKDSIDTCD | (SEQ ID NO: 42) | 2339.54 |
| INF-HA-1-V3/11 | DVLFSVKSPNNKNKEPIDTCD | (SEQ ID NO: 43) | 2363.61 |
| INF-HA-1-V3/12 | DVLFSVKSPNNKNKESIDTCD | (SEQ ID NO: 44) | 2353.57 |
| INF-HA-1-V3/13 | DVLFSVKSVNNKNKDPIDTCD | (SEQ ID NO: 45) | 2351.6 |
| INF-HA-1-V3/14 | DVLFSVKSVNNKNKDSIDTCD | (SEQ ID NO: 46) | 2341.56 |
| INF-HA-1-V3/15 | DVLFSVKSVNNKNKEPIDTCD | (SEQ ID NO: 47) | 2365.62 |
| INF-HA-1-V3/16 | DVLFSVKSVNNKNKESIDTCD | (SEQ ID NO: 48) | 2355.58 |

TABLE 4

Discosite constructs for INF-HA-1-V4
YVSVSTSRIASRPKVRGQSGR (SEQ ID NO: 49)
--T--S---G---W-------

| | | |
|---|---|---|
| Peptide Name | Sequence | MW |

| | | | |
|---|---|---|---|
| INF-HA-1-V4/1 | YVSVSTSRIASRPKVRGQSGR | (SEQ ID NO: 49) | 2291.57 |
| INF-HA-1-V4/2 | YVSVSTSRIASRPWVRGQSGR | (SEQ ID NO: 50) | 2349.61 |
| INF-HA-1-V4/3 | YVSVSTSRIGSRPKVRGQSGR | (SEQ ID NO: 51) | 2277.55 |
| INF-HA-1-V4/4 | YVSVSTSRIGSRPWVRGQSGR | (SEQ ID NO: 52) | 2335.58 |
| INF-HA-1-V4/5 | YVSVSSSRIASRPKVRGQSGR | (SEQ ID NO: 53) | 2277.55 |
| INF-HA-1-V4/6 | YVSVSSSRIASRPWVRGQSGR | (SEQ ID NO: 54) | 2335.58 |
| INF-HA-1-V4/7 | YVSVSSSRIGSRPKVRGQSGR | (SEQ ID NO: 55) | 2263.52 |
| INF-HA-1-V4/8 | YVSVSSSRIGSRPWVRGQSGR | (SEQ ID NO: 56) | 2321.56 |
| INF-HA-1-V4/9 | YVTVSTSRIASRPKVRGQSGR | (SEQ ID NO: 57) | 2305.6 |
| INF-HA-1-V4/10 | YVTVSTSRIASRPWVRGQSGR | (SEQ ID NO: 58) | 2363.64 |
| INF-HA-1-V4/11 | YVTVSTSRIGSRPKVRGQSGR | (SEQ ID NO: 59) | 2291.57 |
| INF-HA-1-V4/12 | YVTVSTSRIGSRPWVRGQSGR | (SEQ ID NO: 60) | 2349.61 |
| INF-HA-1-V4/13 | YVTVSSSRIASRPKVRGQSGR | (SEQ ID NO: 61) | 2291.57 |
| INF-HA-1-V4/14 | YVTVSSSRIASRPWVRGQSGR | (SEQ ID NO: 62) | 2349.61 |
| INF-HA-1-V4/15 | YVTVSSSRIGSRPKVRGQSGR | (SEQ ID NO: 63) | 2277.55 |
| INF-HA-1-V4/16 | YVTVSSSRIGSRPWVRGQSGR | (SEQ ID NO: 64) | 2335.58 |

TABLE 5

Avian influenza A discosite constructs for INF-HA-2-V1
YACKRGGKSSGSSYPVLKVSY (SEQ ID NO: 65)
----Y------------SRT-

| | | |
|---|---|---|
| Peptide Name | Sequence | MW |

| | | | |
|---|---|---|---|
| INF-HA-2-V1/1 | YACKRGGKSSGSSYPVLKVSY | (SEQ ID NO: 65) | 2237.54 |
| INF-HA-2-V1/2 | YACKRGGKSSGSSYPVLKVTY | (SEQ ID NO: 66) | 2251.57 |
| INF-HA-2-V1/3 | YACKRGGKSSGSSYPVLKRSY | (SEQ ID NO: 67) | 2294.6 |
| INF-HA-2-V1/4 | YACKRGGKSSGSSYPVLKRTY | (SEQ ID NO: 68) | 2308.62 |
| INF-HA-2-V1/5 | YACKRGGKSSGSSYPVLSVSY | (SEQ ID NO: 69) | 2196.44 |
| INF-HA-2-V1/6 | YACKRGGKSSGSSYPVLSVTY | (SEQ ID NO: 70) | 2210.47 |
| INF-HA-2-V1/7 | YACKRGGKSSGSSYPVLSRSY | (SEQ ID NO: 71) | 2253.5 |
| INF-HA-2-V1/8 | YACKRGGKSSGSSYPVLSRTY | (SEQ ID NO: 72) | 2267.53 |
| INF-HA-2-V1/9 | YACKYGGKSSGSSYPVLKVSY | (SEQ ID NO: 73) | 2244.53 |
| INF-HA-2-V1/10 | YACKYGGKSSGSSYPVLKVTY | (SEQ ID NO: 74) | 2258.56 |
| INF-HA-2-V1/11 | YACKYGGKSSGSSYPVLKRSY | (SEQ ID NO: 75) | 2301.58 |
| INF-HA-2-V1/12 | YACKYGGKSSGSSYPVLKRTY | (SEQ ID NO: 76) | 2315.61 |
| INF-HA-2-V1/13 | YACKYGGKSSGSSYPVLSVSY | (SEQ ID NO: 77) | 2203.43 |
| INF-HA-2-V1/14 | YACKYGGKSSGSSYPVLSVTY | (SEQ ID NO: 78) | 2217.46 |
| INF-HA-2-V1/15 | YACKYGGKSSGSSYPVLSRSY | (SEQ ID NO: 79) | 2260.49 |
| INF-HA-2-V1/16 | YACKYGGKSSGSSYPVLSRTY | (SEQ ID NO: 80) | 2274.51 |

TABLE 6

Avian influenza A discosite constructs for INF-HA-2-V2
(Discosite B2)
KKGSVHHPSTITEQTSLYVNA (SEQ ID NO: 81)
-S-------------K--QQ-

| | | |
|---|---|---|
| Peptide Name | Sequence | MW |

| | | | |
|---|---|---|---|
| INF-HA-2-V2/1 | KKGSVHHPSTITEQTSLYVNA | (SEQ ID NO: 81) | 2297.53 |
| INF-HA-2-V2/2 | KKGSVHHPSTITEQTSLYVQA | (SEQ ID NO: 82) | 2311.55 |
| INF-HA-2-V2/3 | KKGSVHHPSTITEQTSLYQNA | (SEQ ID NO: 83) | 2326.53 |
| INF-HA-2-V2/4 | KKGSVHHPSTITEQTSLYQQA | (SEQ ID NO: 84) | 2340.55 |
| INF-HA-2-V2/5 | KKGSVHHPSTITEQTKLYVNA | (SEQ ID NO: 85) | 2338.62 |
| INF-HA-2-V2/6 | KKGSVHHPSTITEQTKLYVQA | (SEQ ID NO: 86) | 2352.65 |
| INF-HA-2-V2/7 | KKGSVHHPSTITEQTKLYQNA | (SEQ ID NO: 87) | 2367.62 |
| INF-HA-2-V2/8 | KKGSVHHPSTITEQTKLYQQA | (SEQ ID NO: 88) | 2381.65 |
| INF-HA-2-V2/9 | KSGSVHHPSTITEQTSLYVNA | (SEQ ID NO: 89) | 2256.43 |
| INF-HA-2-V2/10 | KSGSVHHPSTITEQTSLYVQA | (SEQ ID NO: 90) | 2270.46 |
| INF-HA-2-V2/11 | KSGSVHHPSTITEQTSLYQNA | (SEQ ID NO: 91) | 2285.43 |
| INF-HA-2-V2/12 | KSGSVHHPSTITEQTSLYQQA | (SEQ ID NO: 92) | 2299.46 |
| INF-HA-2-V2/13 | KSGSVHHPSTITEQTKLYVNA | (SEQ ID NO: 93) | 2297.53 |
| INF-HA-2-V2/14 | KSGSVHHPSTITEQTKLYVQA | (SEQ ID NO: 94) | 2311.55 |
| INF-HA-2-V2/15 | KSGSVHHPSTITEQTKLYQNA | (SEQ ID NO: 95) | 2326.53 |
| INF-HA-2-V2/16 | KSGSVHHPSTITEQTKLYQQA | (SEQ ID NO: 96) | 2340.55 |

TABLE 7

Avian influenza A discosite constructs for INF-HA-2-V3
DVLFSVESPNNKNKDPIDTCD (SEQ ID NO: 97)
------P-N-----EE-----

| | | |
|---|---|---|
| Peptide Name | Sequence | MW |

| | | | |
|---|---|---|---|
| INF-HA-2-V3/1 | DVLFSVESPNNKNKDPIDTCD | (SEQ ID NO: 97) | 2350.52 |
| INF-HA-2-V3/2 | DVLFSVESPNNKNKDEIDTCD | (SEQ ID NO: 98) | 2382.52 |
| INF-HA-2-V3/3 | DVLFSVESPNNKNKEPIDTCD | (SEQ ID NO: 99) | 2364.55 |
| INF-HA-2-V3/4 | DVLFSVESPNNKNKEEIDTCD | (SEQ ID NO: 100) | 2396.55 |
| INF-HA-2-V3/5 | DVLFSVESNNNKNKDPIDTCD | (SEQ ID NO: 101) | 2367.51 |
| INF-HA-2-V3/6 | DVLFSVESNNNKNKDEIDTCD | (SEQ ID NO: 102) | 2399.51 |
| INF-HA-2-V3/7 | DVLFSVESNNNKNKEPIDTCD | (SEQ ID NO: 103) | 2381.53 |
| INF-HA-2-V3/8 | DVLFSVESNNNKNKEEIDTCD | (SEQ ID NO: 104) | 2413.53 |
| INF-HA-2-V3/9 | DVLFSVPSPNNKNKDPIDTCD | (SEQ ID NO: 105) | 2318.52 |
| INF-HA-2-V3/10 | DVLFSVPSPNNKNKDEIDTCD | (SEQ ID NO: 106) | 2350.52 |
| INF-HA-2-V3/11 | DVLFSVPSPNNKNKEPIDTCD | (SEQ ID NO: 107) | 2332.55 |
| INF-HA-2-V3/12 | DVLFSVPSPNNKNKEEIDTCD | (SEQ ID NO: 108) | 2364.55 |
| INF-HA-2-V3/13 | DVLFSVPSNNNKNKDPIDTCD | (SEQ ID NO: 109) | 2335.51 |
| INF-HA-2-V3/14 | DVLFSVPSNNNKNKDEIDTCD | (SEQ ID NO: 110) | 2367.51 |
| INF-HA-2-V3/15 | DVLFSVPSNNNKNKEPIDTCD | (SEQ ID NO: 111) | 2349.54 |
| INF-HA-2-V3/16 | DVLFSVPSNNNKNKEEIDTCD | (SEQ ID NO: 112) | 2381.53 |

TABLE 8

Avian influenza A discosite constructs for INF-HA-2-V4
YVSVSTSRIASRPKVRGQSGR (SEQ ID NO: 113)
--T--S---G---W-------

| | | |
|---|---|---|
| Peptide Name | Sequence | NW |

| | | | |
|---|---|---|---|
| INF-HA-2-V4/1 | YVSVSTSRIASRPKVRGQSGR | (SEQ ID NO: 113) | 2291.57 |
| INF-HA-2-V4/2 | YVSVSTSRIASRPWVRGQSGR | (SEQ ID NO: 114) | 2349.61 |
| INF-HA-2-V4/3 | YVSVSTSRIGSRPKVRGQSGR | (SEQ ID NO: 115) | 2277.55 |
| INF-HA-2-V4/4 | YVSVSTSRIGSRPNVRGQSGR | (SEQ ID NO: 116) | 2335.58 |
| INF-HA-2-V4/5 | YVSVSSSRIASRPKVRGQSGR | (SEQ ID NO: 117) | 2277.55 |
| INF-HA-2-V4/6 | YVSVSSSRIASRPNVRGQSGR | (SEQ ID NO: 118) | 2335.58 |
| INF-HA-2-V4/7 | YVSVSSSRIGSRPKVRGQSGR | (SEQ ID NO: 119) | 2263.52 |
| INF-HA-2-V4/8 | YVSVSSSRIGSRPWVRGQSGR | (SEQ ID NO: 120) | 2321.56 |
| INF-HA-2-V4/9 | YVTVSTSRIASRPKVRGQSGR | (SEQ ID NO: 121) | 2305.6 |
| INF-HA-2-V4/10 | YVTVSTSRIASRPWVRGQSGR | (SEQ ID NO: 122) | 2363.64 |
| INF-HA-2-V4/11 | YVTVSTSRIGSRPKVRGQSGR | (SEQ ID NO: 123) | 2291.57 |
| INF-HA-2-V4/12 | YVTVSTSRIGSRPWVRGQSGR | (SEQ ID NO: 124) | 2349.61 |
| INF-HA-2-V4/13 | YVTVSSSRIASRPKVRGQSGR | (SEQ ID NO: 125) | 2291.57 |
| INF-HA-2-V4/14 | YVTVSSSRIASRPWVRGQSGR | (SEQ ID NO: 126) | 2349.61 |
| INF-HA-2-V4/15 | YVTVSSSRIGSRPKVRGQSGR | (SEQ ID NO: 127) | 2277.55 |
| INF-HA-2-V4/16 | YVTVSSSRIGSRPWVRGQSGR | (SEQ ID NO: 128) | 2335.58 |

TABLE 9

Human influenza B discosite constructs for INF-HB-1-V1
GSCPNATNRNGDNNKTAINPLTVEVPY (SEQ ID NO: 133)
-------S-S-------T---------

| | | |
|---|---|---|
| Peptide Name | Sequence | MW |

| | | | |
|---|---|---|---|
| INF-HB-1-V1/1 | GSCPNATNRNGDNNKTAINPLTVEVPY | (SEQ ID NO: 133) | 2860.08 |
| INF-HB-1-V1/2 | GSCPNATNRNGDNNKTATNPLTVEVPY | (SEQ ID NO: 134) | 2848.03 |
| INF-HB-1-V1/3 | GSCPNATNRSGDNNKTAINPLTVEVPY | (SEQ ID NO: 135) | 2833.06 |
| INF-HB-1-V1/4 | GSCPNATNRSGDNNKTATNPLTVEVPY | (SEQ ID NO: 136) | 2821 |
| INF-HB-1-V1/5 | GSCPNATSRNGDNNKTAINPLTVEVPY | (SEQ ID NO: 137) | 2833.06 |
| INF-HB-1-V1/6 | GSCPNATSRNGDNNKTATNPLTVEVPY | (SEQ ID NO: 138) | 2821 |
| INF-HB-1-V1/7 | GSCPNATSRSGDNNKTAINPLTVEVPY | (SEQ ID NO: 139) | 2806.03 |
| INF-HB-1-V1/8 | GSCPNATSRSGDNNKTATNPLTVEVPY | (SEQ ID NO: 140) | 2793.98 |

TABLE 10

Human influenza B discosite constructs for INF-HB-1-V2
PKDNFHSDNKTQMERLYGDSN (SEQ ID NO: 141)
RN-----------KN------

| | | |
|---|---|---|
| Peptide Name | Sequence | MW |

| | | | |
|---|---|---|---|
| INF-HB-1-V2/1 | PKDNFHSDNKTQMERLYGDSN | (SEQ ID NO: 141) | 2496.63 |
| INF-HB-1-V2/2 | PKDNFHSDNKTQMENLYGDSN | (SEQ ID NO: 142) | 2454.55 |
| INF-HB-1-V2/3 | PKDNFHSDNKTQMKRLYGDSN | (SEQ ID NO: 143) | 2495.69 |
| INF-HB-1-V2/4 | PKDNFHSDNKTQMKNLYGDSN | (SEQ ID NO: 144) | 2453.61 |
| INF-HB-1-V2/5 | PNDNFHSDNKTQMERLYGDSN | (SEQ ID NO: 145) | 2482.56 |
| INF-HB-1-V2/6 | PNDNFHSDNKTQMENLYGDSN | (SEQ ID NO: 146) | 2440.48 |
| INF-HB-1-V2/7 | PNDNFHSDNKTQMKRLYGDSN | (SEQ ID NO: 147) | 2481.62 |
| INF-HB-1-V2/8 | PNDNFHSDNKTQMKNLYGDSN | (SEQ ID NO: 148) | 2439.54 |
| INF-HB-1-V2/9 | RKDNFHSDNKTQMERLYGDSN | (SEQ ID NO: 149) | 2555.7 |
| INF-HB-1-V2/10 | RKDNFHSDNKTQMENLYGDSN | (SEQ ID NO: 150) | 2513.62 |
| INF-HB-1-V2/11 | RKDNFHSDNKTQMKRLYGDSN | (SEQ ID NO: 151) | 2554.76 |
| INF-HB-1-V2/12 | RKDNFHSDNKTQMKNLYGDSN | (SEQ ID NO: 152) | 2512.68 |
| INF-HB-1-V2/13 | RNDNFHSDNKTQMERLYGDSN | (SEQ ID NO: 153) | 2541.63 |
| INF-HB-1-V2/14 | RNDNFHSDNKTQMENLYGDSN | (SEQ ID NO: 154) | 2499.55 |
| INF-HB-1-V2/15 | RNDNFHSDNKTQMKRLYGDSN | (SEQ ID NO: 155) | 2540.69 |
| INF-HB-1-V2/16 | RNDNFHSDNKTQMKNLYGDSN | (SEQ ID NO: 156) | 2498.61 |

TABLE 11

Human influenza discosite constructs for INF-HB-1-V3
RGKLCPNCFNCTDIICSEGEDLPLIGE (SEQ ID NO: 157)
--------L----L--TK---------

| | | |
|---|---|---|
| Peptide Name | Sequence | MW |

| | | | |
|---|---|---|---|
| INF-HB-1-V3/1 | RGKLCPNCFNCTDIICSEGEDLPLIGE | (SEQ ID NO: 157) | 2940 |
| INF-HB-1-V3/2 | RGKLCPNCFNCTDIICSKGEDLPLIGE | (SEQ ID NO: 158) | 2939 |
| INF-HB-1-V3/3 | RGKLCPNCFNCTDIICTEGEDLPLIGE | (SEQ ID NO: 159) | 2954 |
| INF-HB-1-V3/4 | RGKLCPNCFNCTDIICTKGEDLPLIGE | (SEQ ID NO: 160) | 2953 |
| INF-HB-1-V3/5 | RGKLCPNCFNCTDLICSEGEDLPLIGE | (SEQ ID NO: 161) | 2940 |
| INF-HB-1-V3/6 | RGKLCPNCFNCTDLICSKGEDLPLIGE | (SEQ ID NO: 162) | 2939 |
| INF-HB-1-V3/7 | RGKLCPNCFNCTDLICTEGEDLPLIGE | (SEQ ID NO: 163) | 2954 |
| INF-HB-1-V3/8 | RGKLCPNCFNCTDLICTKGEDLPLIGE | (SEQ ID NO: 164) | 2953 |
| INF-HB-1-V3/9 | RGKLCPNCLNCTDIICSEGEDLPLIGE | (SEQ ID NO: 165) | 2906 |
| INF-HB-1-V3/10 | RGKLCPNCLNCTDIICSKGEDLPLIGE | (SEQ ID NO: 166) | 2905 |
| INF-HB-1-V3/11 | RGKLCPNCLNCTDIICTEGEDLPLIGE | (SEQ ID NO: 167) | 2920 |
| INF-HB-1-V3/12 | RGKLCPNCLNCTDIICTKGEDLPLIGE | (SEQ ID NO: 168) | 2919 |
| INF-HB-1-V3/13 | RGKLCPNCLNCTDLICSEGEDLPLIGE | (SEQ ID NO: 169) | 2906 |
| INF-HB-1-V3/14 | RGKLCPNCLNCTDLICSKGEDLPLIGE | (SEQ ID NO: 170) | 2905 |
| INF-HB-1-V3/15 | RGKLCPNCLNCTDLICTEGEDLPLIGE | (SEQ ID NO: 171) | 2920 |
| INF-HB-1-V3/16 | RGKLCPNCLNCTDLICTKGEDLPLIGE | (SEQ ID NO: 172) | 2919 |

TABLE 12

Human Influenza B discosite constructs for INF-HB-1-V4
KFTSSANGIGGFPNQTEDEGLKQSGR (SEQ ID NO: 173)
-------------D----G--P----

| | | |
|---|---|---|
| Peptide Name | Sequence | MW |

| | | | |
|---|---|---|---|
| INF-HB-1-V4/1 | KFTSSANGIGGFPNQTEDEGLKQSGR | (SEQ ID NO: 173) | 2725.88 |
| INF-HB-1-V4/2 | KFTSSANGIGGFPNQTEDEGLPQSGR | (SEQ ID NO: 174) | 2694.82 |
| INF-HB-1-V4/3 | KFTSSANGIGGFPNQTEDGGLKQSGR | (SEQ ID NO: 175) | 2653.82 |
| INF-HB-1-V4/4 | KFTSSANGIGGFPNQTEDGGLPQSGR | (SEQ ID NO: 176) | 2622.76 |
| INF-HB-1-V4/5 | KFTSSANGIGGFPDQTEDEGLKQSGR | (SEQ ID NO: 177) | 2726.87 |
| INF-HB-1-V4/6 | KFTSSANGIGGFPDQTEDEGLPQSGR | (SEQ ID NO: 178) | 2695.81 |
| INF-HB-1-V4/7 | KFTSSANGIGGFPDQTEDGGLKQSGR | (SEQ ID NO: 179) | 2654.8 |
| INF-HB-1-V4/8 | KFTSSANGIGGFPDQTEDGGLPQSGR | (SEQ ID NO: 180) | 2623.75 |

TABLE 13

Equine influenza discosite constructs for INFE-HA-1-V1
SACKRRSASSNAAFPQMNKTM (SEQ ID NO: 185)
-------------Y---T-SY

| | | |
|---|---|---|
| Peptide Name | Sequence | MW |

| | | | |
|---|---|---|---|
| INFE-HA-1-V1/1 | SACKRRSASSNAAFPQMNKTM | (SEQ ID NO: 185) | 2286.62 |
| INFE-HA-1-V1/2 | SACKRRSASSNAAFPQMNKTY | (SEQ ID NO: 186) | 2318.6 |
| INFE-HA-1-V1/3 | SACKRRSASSNAAFPQMNKSM | (SEQ ID NO: 187) | 2272.6 |
| INFE-HA-1-V1/4 | SACKRRSASSNAAFPQMNKSY | (SEQ ID NO: 188) | 2304.57 |
| INFE-HA-1-V1/5 | SACKRRSASSNAAFPQMTKTM | (SEQ ID NO: 189) | 2273.62 |
| INFE-HA-1-V1/6 | SACKRRSASSNAAFPQMTKTY | (SEQ ID NO: 190) | 2305.6 |
| INFE-HA-1-V1/7 | SACKRRSASSNAAFPQMTKSM | (SEQ ID NO: 191) | 2259.6 |
| INFE-HA-1-V1/8 | SACKRRSASSNAAFPQMTKSY | (SEQ ID NO: 192) | 2291.57 |
| INFE-HA-1-V1/9 | SACKRRSASSNAAYPQMNKTM | (SEQ ID NO: 193) | 2302.62 |
| INFE-HA-1-V1/10 | SACKRRSASSNAAYPQMNKTY | (SEQ ID NO: 194) | 2334.6 |
| INFE-HA-1-V1/11 | SACKRRSASSNAAYPQMNKSM | (SEQ ID NO: 195) | 2288.6 |
| INFE-HA-1-V1/12 | SACKRRSASSNAAYPQMNKSY | (SEQ ID NO: 196) | 2320.57 |
| INFE-HA-1-V1/13 | SACKRRSASSNAAYPQMTKTM | (SEQ ID NO: 197) | 2289.62 |
| INFE-HA-1-V1/14 | SACKRRSASSNAAYPQMTKTY | (SEQ ID NO: 198) | 2321.6 |
| INFE-HA-1-V1/15 | SACKRRSASSNAAYPQMTKSM | (SEQ ID NO: 199) | 2275.6 |
| INFE-HA-1-V1/16 | SACKRRSASSNAAYPQMTKSY | (SEQ ID NO: 200) | 2307.57 |

TABLE 14

Equine influenza discosite constructs for INFE-HA-1-V2
SSTDNAIHHSSSNQEQTKLYVQE (SEQ ID NO: 201)
-N-------P---T-------S-

| | | |
|---|---|---|
| Peptide Name | Sequence | MW |

| | | | |
|---|---|---|---|
| INFE-HA-1-V2/1 | SSTDNAIHHSSSNQEQTKLYVQE | (SEQ ID NO: 201) | 2603.67 |
| INFE-HA-1-V2/2 | SSTDNAIHHSSSNQEQTKLYVSE | (SEQ ID NO: 202) | 2562.62 |
| INFE-HA-1-V2/3 | SSTDNAIHHSSSNTEQTKLYVQE | (SEQ ID NO: 203) | 2576.64 |
| INFE-HA-1-V2/4 | SSTDNAIHHSSSNTEQTKLYVSE | (SEQ ID NO: 204) | 2535.59 |
| INFE-HA-1-V2/5 | SSTDNAIHHPSSNQEQTKLYVQE | (SEQ ID NO: 205) | 2613.71 |
| INFE-HA-1-V2/6 | SSTDNAIHHPSSNQEQTKLYVSE | (SEQ ID NO: 206) | 2572.66 |
| INFE-HA-1-V2/7 | SSTDNAIHHPSSNTEQTKLYVQE | (SEQ ID NO: 207) | 2586.68 |
| INFE-HA-1-V2/8 | SSTDNAIHHPSSNTEQTKLYVSE | (SEQ ID NO: 208) | 2545.63 |
| INFE-HA-1-V2/9 | SNTDNAIHHSSSNQEQTKLYVQE | (SEQ ID NO: 209) | 2630.69 |
| INFE-HA-1-V2/10 | SNTDNAIHHSSSNQEQTKLYVSE | (SEQ ID NO: 210) | 2589.64 |
| INFE-HA-1-V2/11 | SNTDNAIHHSSSNTEQTKLYVQE | (SEQ ID NO: 211) | 2603.67 |
| INFE-HA-1-V2/12 | SNTDNAIHHSSSNTEQTKLYVSE | (SEQ ID NO: 212) | 2562.62 |
| INFE-HA-1-V2/13 | SNTDNAIHHPSSNQEQTKLYVQE | (SEQ ID NO: 213) | 2640.73 |
| INFE-HA-1-V2/14 | SNTDNAIHHPSSNQEQTKLYVSE | (SEQ ID NO: 214) | 2599.68 |
| INFE-HA-1-V2/15 | SNTDNAIHHPSSNTEQTKLYVQE | (SEQ ID NO: 215) | 2613.71 |
| INFE-HA-1-V2/16 | SNTDNAIHHPSSNTEQTKLYVSE | (SEQ ID NO: 216) | 2572.66 |

TABLE 15

Equine influenza discosite constructs for INFE-HA-1-V3
DQFQEESPNNRNFDPDDNCE (SEQ ID NO: 217)
---L-F---T---P------

| | | |
|---|---|---|
| Peptide Name | Sequence | MW |

| | | | |
|---|---|---|---|
| INFE-HA-1-V3/1 | DQFQEESPNNRNFDPDDNCE | (SEQ ID NO: 217) | 2413.36 |
| INFE-HA-1-V3/2 | DQFQEESPNNRNFPPDDNCE | (SEQ ID NO: 218) | 2395.39 |
| INFE-HA-1-V3/3 | DQFQEESPNTRNFDPDDNCE | (SEQ ID NO: 219) | 2400.36 |
| INFE-HA-1-V3/4 | DQFQEESPNTRNFPPDDNCE | (SEQ ID NO: 220) | 2382.39 |
| INFE-HA-1-V3/5 | DQFQEFSPNNRNFDPDDNCE | (SEQ ID NO: 221) | 2431.42 |
| INFE-HA-1-V3/6 | DQFQEFSPNNRNFPPDDNCE | (SEQ ID NO: 222) | 2413.45 |
| INFE-HA-1-V3/7 | DQFQEFSPNTRNFDPDDNCE | (SEQ ID NO: 223) | 2418.42 |
| INFE-HA-1-V3/8 | DQFQEFSPNTRNFPPDDNCE | (SEQ ID NO: 224) | 2400.45 |
| INFE-HA-1-V3/9 | DQFLEESPNNRNFDPDDNCE | (SEQ ID NO: 225) | 2398.39 |
| INFE-HA-1-V3/10 | DQFLEESPNNRNFPPDDNCE | (SEQ ID NO: 226) | 2380.42 |
| INFE-HA-1-V3/11 | DQFLEESPNTRNFDPDDNCE | (SEQ ID NO: 227) | 2385.39 |
| INFE-HA-1-V3/12 | DQFLEESPNTRNFPPDDNCE | (SEQ ID NO: 228) | 2367.42 |
| INFE-HA-1-V3/13 | DQFLEFSPNNRNFDPDDNCE | (SEQ ID NO: 229) | 2416.45 |
| INFE-HA-1-V3/14 | DQFLEFSPNNRNFPPDDNCE | (SEQ ID NO: 230) | 2398.48 |
| INFE-HA-1-V3/15 | DQFLEFSPNTRNFDPDDNCE | (SEQ ID NO: 231) | 2403.45 |
| INFE-HA-1-V3/16 | DQFLEFSPNTRNFPPDDNCE | (SEQ ID NO: 232) | 2385.48 |

TABLE 16

Equine influenza discosite constructs for INFE-HA-1-V4
RITVSTSRPGARPWVRGQSGR (SEQ ID NO: 233)
-----S----S--Q-N-----

| | | |
|---|---|---|
| Peptide Name | Sequence | MW |

| | | | |
|---|---|---|---|
| INFE-HA-1-V4/1 | RITVSTSRPGARPWVRGQSGR | (SEQ ID NO: 233) | 2324.61 |
| INFE-HA-1-V4/2 | RITVSTSRPGARPWVNGQSGR | (SEQ ID NO: 234) | 2282.52 |
| INFE-HA-1-V4/3 | RITVSTSRPGARPQVRGQSGR | (SEQ ID NO: 235) | 2266.53 |
| INFE-HA-1-V4/4 | RITVSTSRPGARPQVNGQSGR | (SEQ ID NO: 236) | 2224.44 |
| INFE-HA-1-V4/5 | RITVSTSRPGSRPNVRGQSGR | (SEQ ID NO: 237) | 2340.61 |
| INFE-HA-1-V4/6 | RITVSTSRPGSRPWVNGQSGR | (SEQ ID NO: 238) | 2298.52 |
| INFE-HA-1-V4/7 | RITVSTSRPGSRPQVRGOSGR | (SEQ ID NO: 239) | 2282.52 |
| INFE-HA-1-V4/8 | RITVSTSRPGSRPQVNGQSGR | (SEQ ID NO: 240) | 2240.44 |
| INFE-HA-1-V4/9 | RITVSSSRPGARPWVRGQSGR | (SEQ ID NO: 241) | 2310.58 |
| INFE-HA-1-V4/10 | RITVSSSRPGARPWVNGQSGR | (SEQ ID NO: 242) | 2268.5 |
| INFE-HA-1-V4/11 | RITVSSSRPGARPQVRGQSGR | (SEQ ID NO: 243) | 2252.5 |
| INFE-HA-1-V4/12 | RITVSSSRPGARPQVNGQSGR | (SEQ ID NO: 244) | 2210.42 |
| INFE-HA-1-V4/13 | RITVSSSRPGSRPWVRGQSGR | (SEQ ID NO: 245) | 2326.58 |
| INFE-HA-1-V4/14 | RITVSSSRPGSRPWVNGQSGR | (SEQ ID NO: 246) | 2284.5 |
| INFE-HA-1-V4/15 | RITVSSSRPGSRPQVRGQSGR | (SEQ ID NO: 247) | 2268.5 |
| INFE-HA-1-V4/16 | RITVSSSRPGSRPQVNGQSGR | (SEQ ID NO: 248) | 2226.42 |

TABLE 17

Influenza A Discotope constructs

| | | |
|---|---|---|
| Peptide Name | Sequence | MW |

| | | | |
|---|---|---|---|
| INF-HA-1-M1 | YACKRGGKSSGSSYPVLNVSY | (SEQ ID NO: 129) | 2223.47 |
| INF-HA-1-M2 | KKGSVHHPSTITEQTSLYVNA | (SEQ ID NO: 130) | 2297.53 |
| INF-HA-1-M3 | DVLFSVESPNNKNKDPIDTCD | (SEQ ID NO: 131) | 2350.52 |
| INF-HA-1-M4 | YVSVSTSRIASRPKVRGQSGR | (SEQ ID NO: 132) | 2291.57 |

TABLE 18

Influenza B Discotope constructs

| | | |
|---|---|---|
| Peptide Name | Sequence | MW |

| | | | |
|---|---|---|---|
| INF-HB-1-M1 | GSCPNVANGNGDNNKTAINPVTVEVPY | (SEQ ID NO: 181) | 2744.95 |
| INF-HB-1-M2 | PKDNFHSDDKTQMERLYGDSN | (SEQ ID NO: 182) | 2497.61 |
| INF-HB-1-M3 | RGKLCPNCFNCTDIICSEGEDLPLIGE | (SEQ ID NO: 183) | 2940.36 |
| INF-HB-1-M4 | KFTSSANGIGGFPNQTEDEGLKQSGR | (SEQ ID NO: 184) | 2725.88 |

### Peptide Synthesis

Peptides were synthesized using standard solid-phase peptide chemistry. The peptides were synthesized by solid phase peptide synthesis (SPPS) using 9-fluoroenylmethoxycarbonyl (Fmoc) chemistry on Pioneer™ automated peptide synthesizer, utilizing pre-loaded Fmoc protected NovaSyn™ TGT resin (NovaBiochem) as described. Where variability at a given position is desired, mixture of two amino acids is placed at that position. This is repeated each time during the synthesis wherever the variability is desired. While 1M solution of 2-(1H-Benzotriazole-1-yl)-1,1,3,3-tetramethyluronium tetrafluoroborate (TBTU) and N-Hydroxybenzotriazole (HOBt) in dimethylformamide (DMF), and 1 M solution of diisopropylethyl amine (DIPEA) in DMF was used for coupling amino acids, 20% piperidine in DMF was used for deblocking amino acids during the synthesis.

Coupling was allowed to occur for one hour at room temperature. After the last amino acid was coupled, the resin was taken out from synthesizer and washed on a sintered glass funnel several times with DMF, with 2-propanol and with dichloromethylene (DCM), and dried under high vacuum. The peptide mixtures are cleaved and deprotected by the addition of a solution containing TFA/water/phenol/thioanisole/EDT/TIS [82:5:5:5:2:1]. The resin was incubated at room temperature for 4 hours. Cleavage mixture was then filtered under reduced pressure into a flask containing a 10-fold volume of cold ether. Resin was also rinsed twice with TFA into the same ether solution.

Following incubation for 30 minutes in a freezer to further assist precipitation, the sample was centrifuged at 1,000×g for 5 minutes, and the ether removed. This extraction process was repeated three times. Following a final ether extraction, the residual organic solvent was evaporated under nitrogen gas, and the peptide mixture was re-dissolved in water and purified by using high performance liquid chromatography (HPLC). Excess of the solvent was removed by using a rotor evaporator, and finally lyophilized to dry powder. Mass spectrometry (MS) and amino acid analysis were performed on all the discotope constructs to ensure that they have the appropriate peptide content.

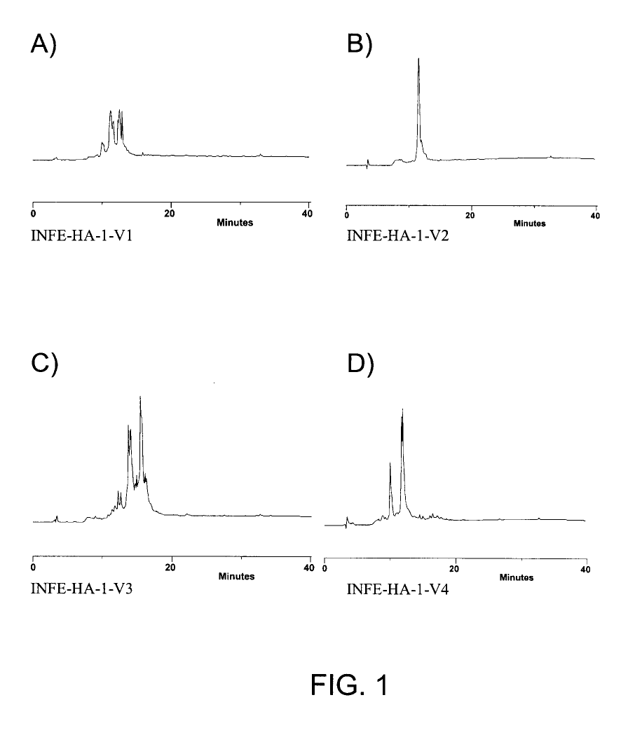

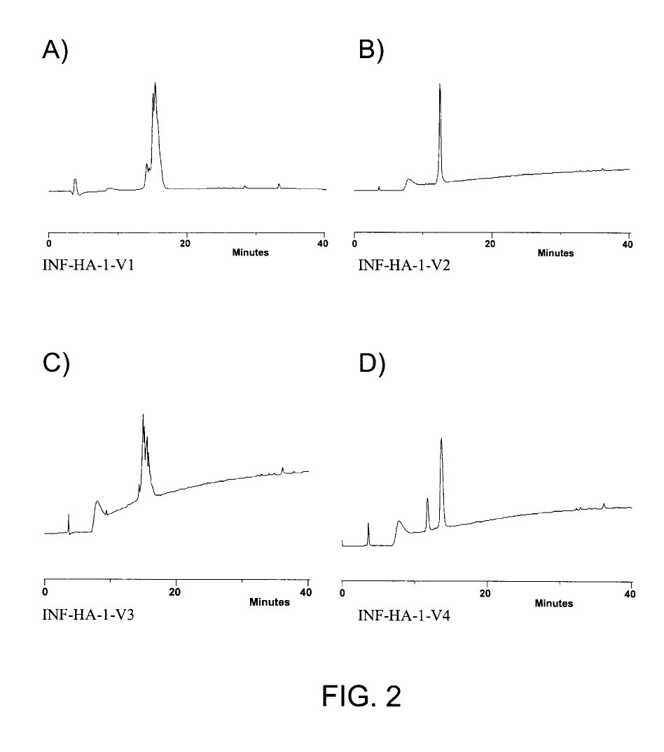

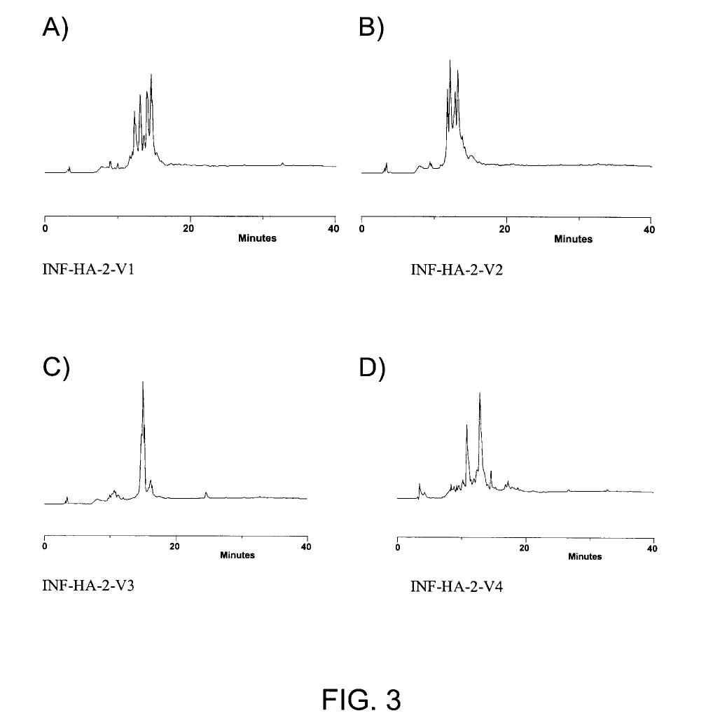

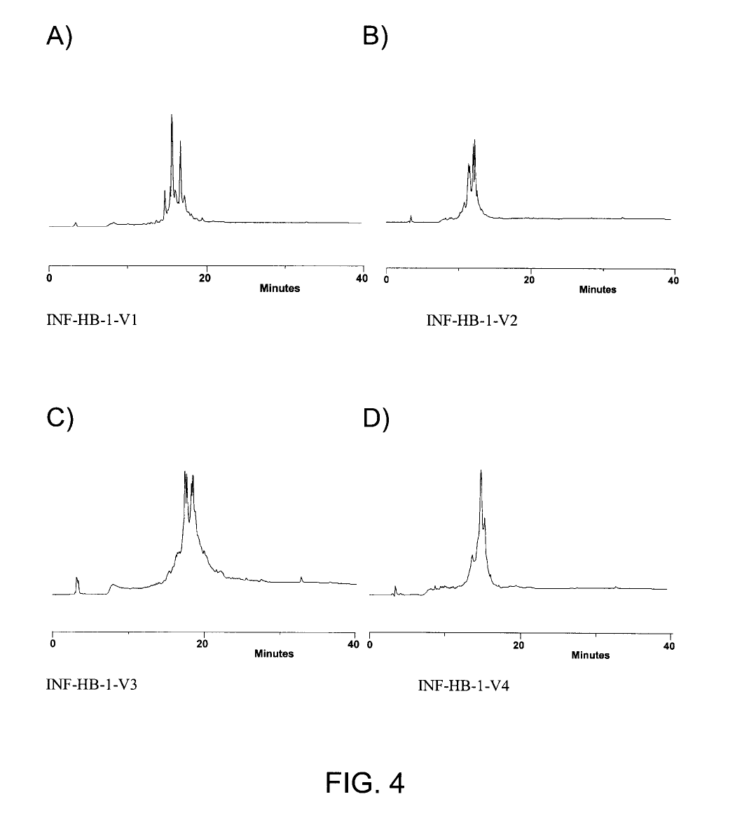

FIGS. 1 to 4 illustrate exemplary HPLC data from the discosite constructs of the present invention. Each HPLC plot corresponds to a particular discosite construct formulation, containing a cocktail of peptides in the respective discosite construct. FIGS. 1A to 1D correspond to discosite constructs INFE-HA-1-V1 to V4 (SEQ ID NOs: 185 to 248), respectively. FIGS. 2A to 2D correspond to discosite constructs INF-HA-1-V1 to V4 (SEQ ID NOs. 1 to 64), respectively. FIGS. 3A to 3D correspond to discosite constructs INF-HA-2-V1 to V4 (SEQ ID NOs: 65 to 128), respectively. FIGS. 4A to 4D correspond to discosite constructs INF-HB-1-V1 to V4 (SEQ ID NOs: 133 to 180), respectively.

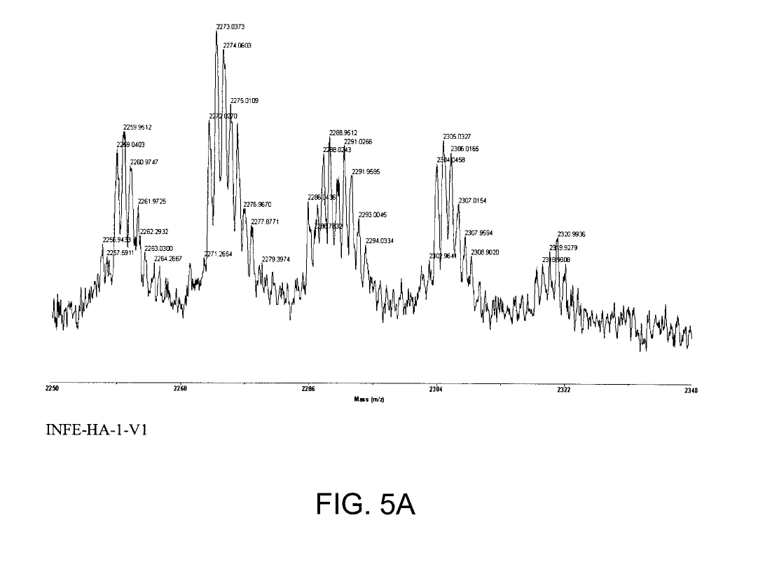

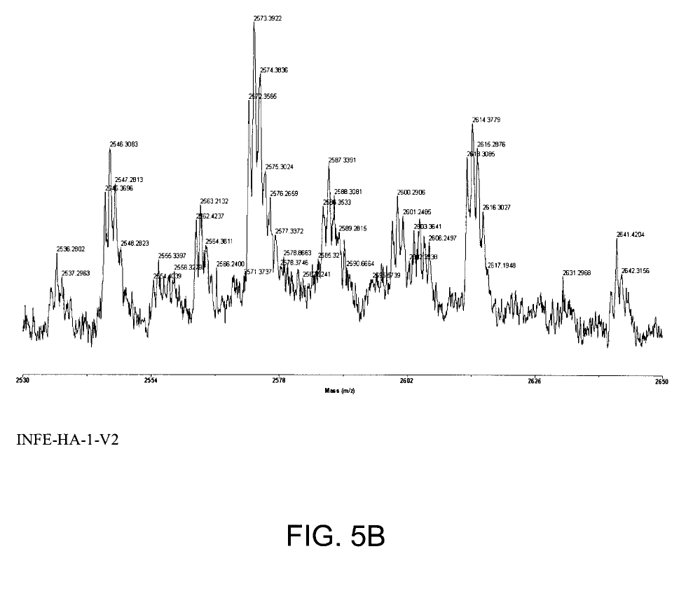

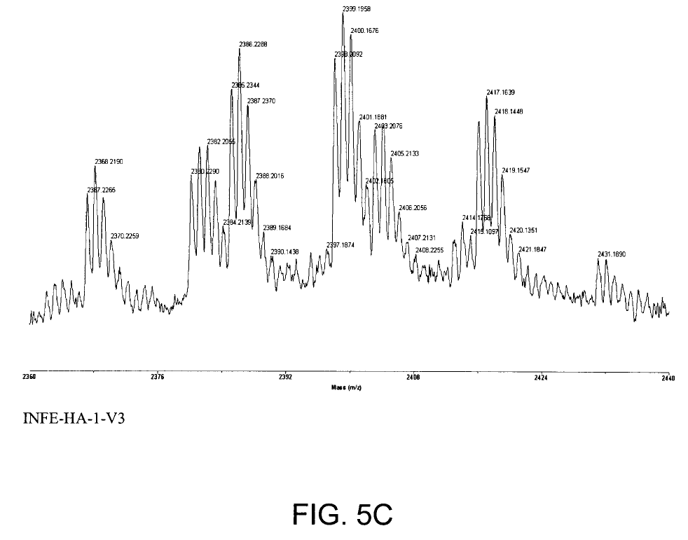

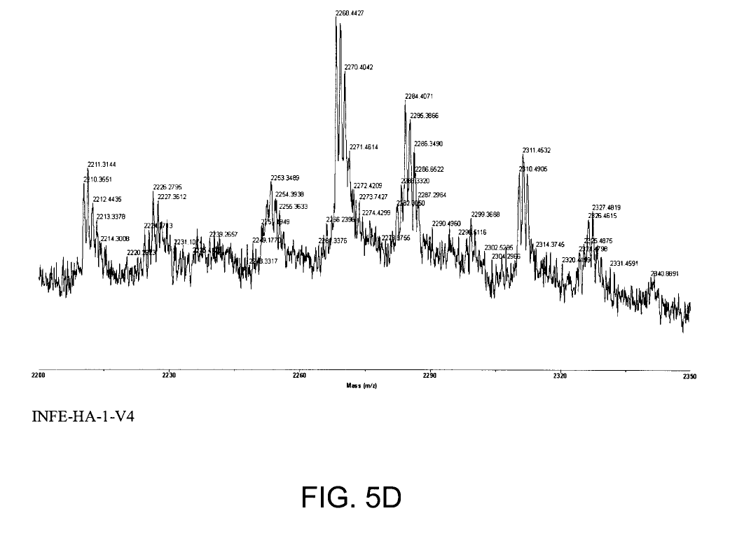

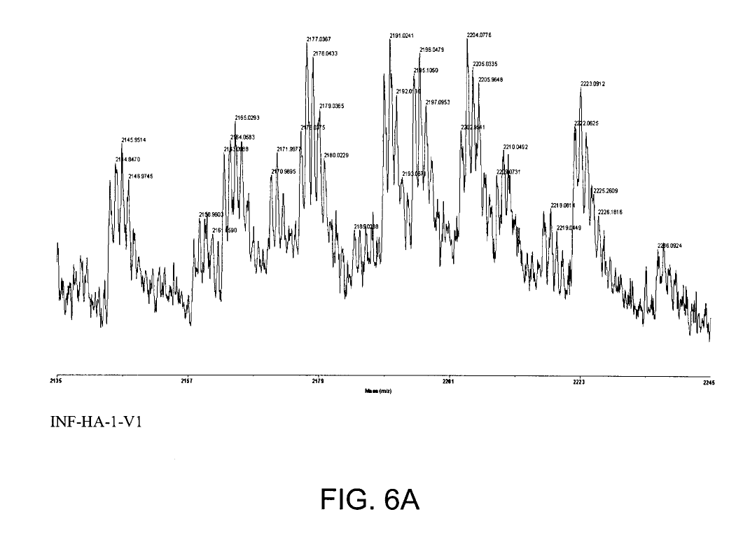

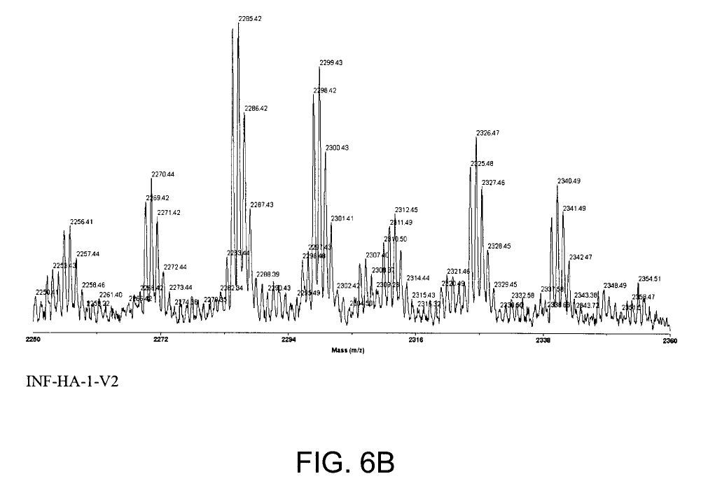

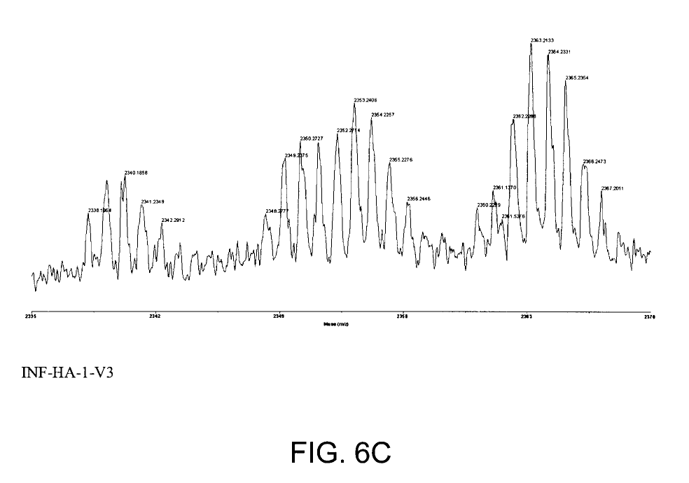

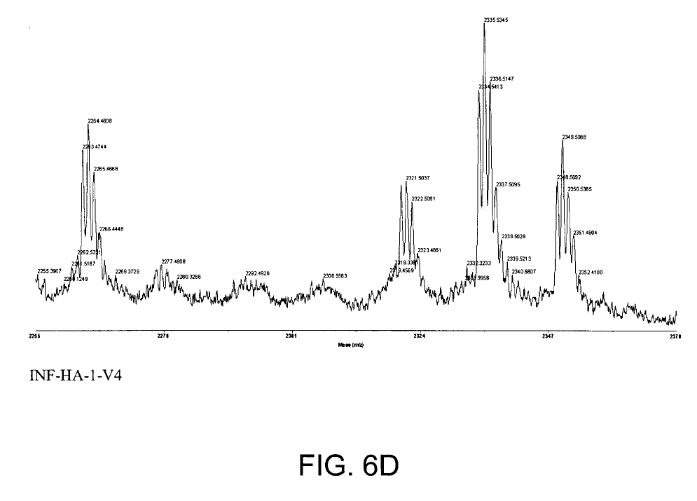

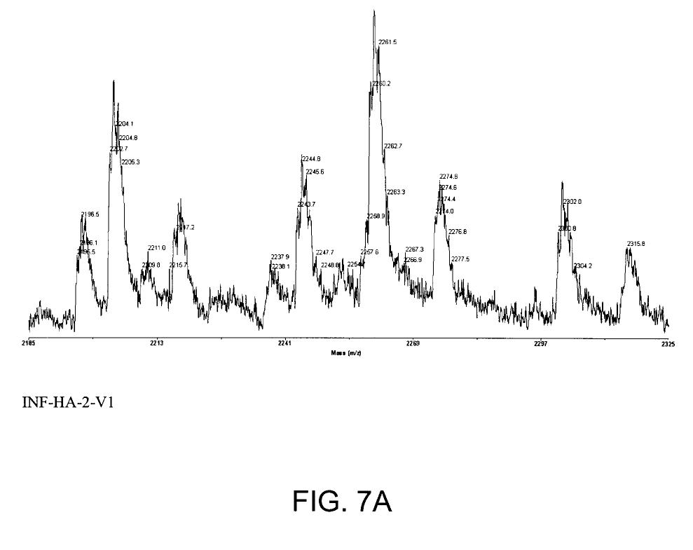

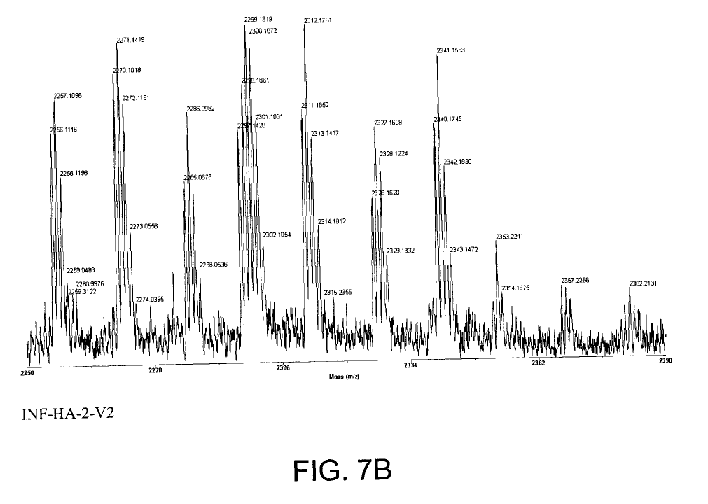

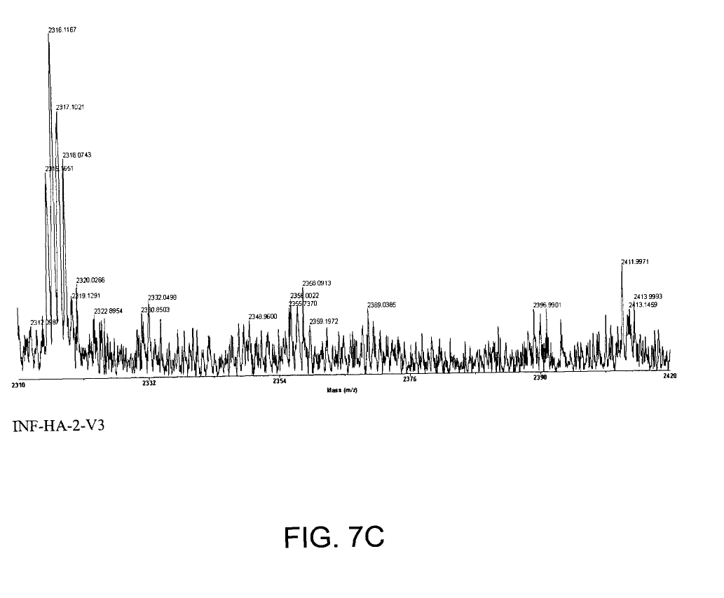

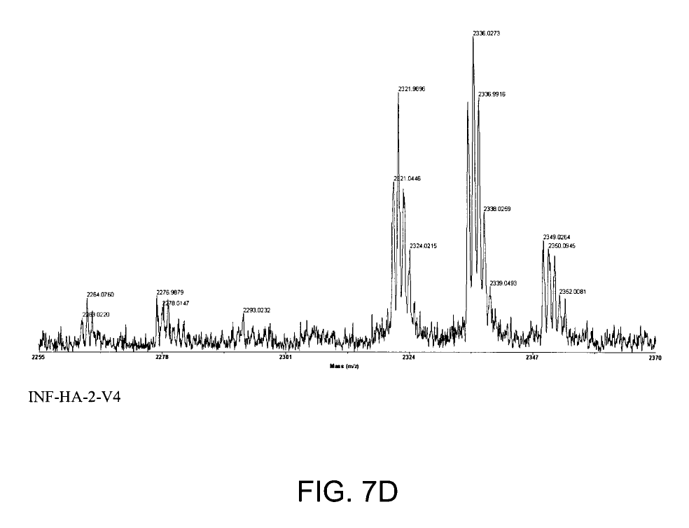

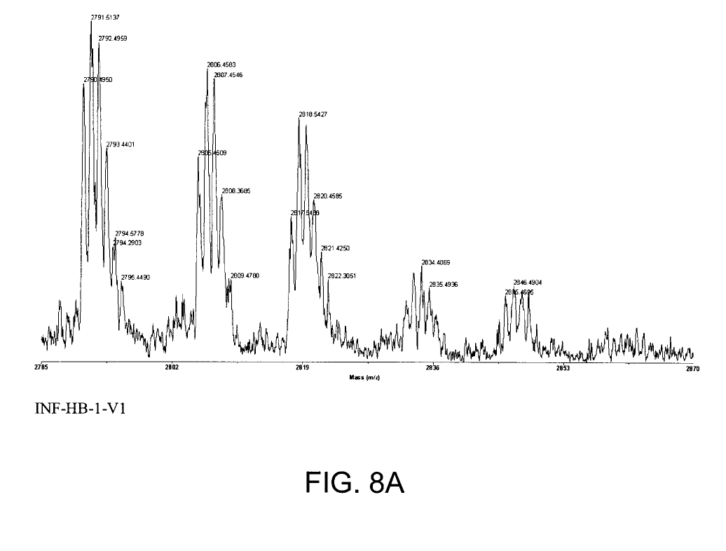

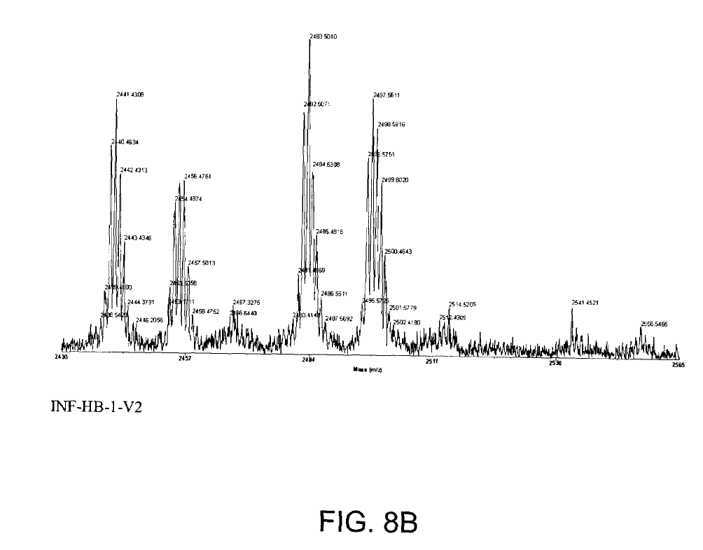

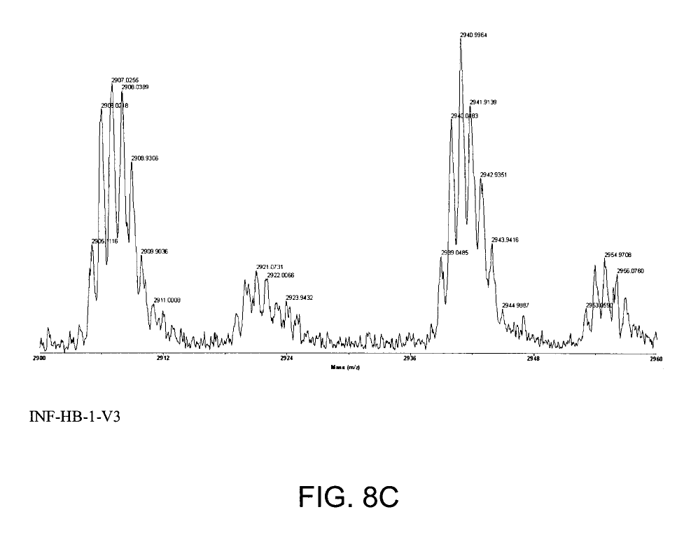

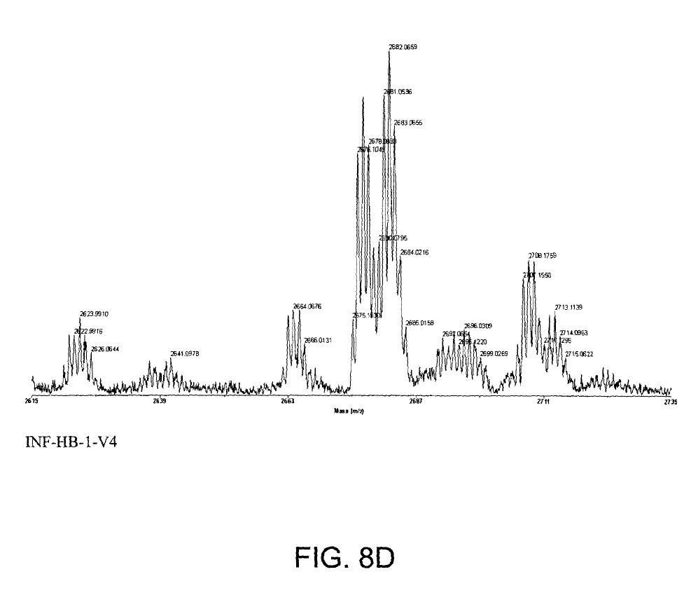

FIGS. 5 to 8 illustrate MS data from the discosite constructs of the present invention. As with the HPLC plots, each MS plot corresponds to a particular discosite construct formulation containing a cocktail of peptides in the respective discosite construct. FIGS. 5A to 5D correspond to discosite constructs INFE-HA-1-V1 to V4 (SEQ ID NOs: 185 to 248), respectively. FIGS. 6A to 6D correspond to discosite constructs INF-HA-1-V1 to V4 (SEQ ID NOs: 1 to 64), respectively. FIGS. 7A to 7D correspond to discosite constructs INF-HA-2-V1 to V4 (SEQ ID NOs: 65 to 128), respectively. FIGS. 8A to 8D correspond to discosite constructs INF-HB-1-V1 to V4 (SEQ ID NOs: 133 to 180), respectively.

### EXAMPLES

Two exemplary vaccines, INF-01P (INF-HA-1-V1-V4) and INFE-01P (INFE-HA-V1-V4), were prepared in accordance with the present invention. The vaccines were prepared based on formulations comprising peptide sequences derived from epitopes from influenza A and as listed in Tables 1 to 4 (SEQ ID NOs: 1 to 64) and Tables 13 to 16 (SEQ ID NOs: 185 to 248), and summarized in Tables 19 and 20, below. Using a murine model, the vaccines were tested: a) to determine whether addition of adjuvant enhances immunity against challenge; b) to determine the humoral response induced by candidate vaccines in comparison to commercial vaccine in a murine model; and c) to assess the range of protection elicited by the vaccine against influenza challenge using different influenza virus strains.

Vaccination schedule: Groups of mice were vaccinated subcutaneously at the base of the tail; mice receiving the commercial vaccine were immunized intramuscularly (as recommended). Mice were similarly boosted two additional times, at three week intervals. Two weeks after the last immunization, the mice were challenged with a lethal dose of H3N2. Mice were monitored daily after challenge for weight and signs of infection.

Adjuvants: The following adjuvants were used to boost immune responses in combination with the vaccine: Ribi (Cedarlane, 1:1 ratio Ribi:vaccine), Alum (Sigma, equal volumes of 500 ng/ml and vaccine), and Montanide (Seppic, 1:1 ratio montanide:vaccine). In the present example, Alum was used as an adjuvant, although any suitable adjuvant can be used.

### Example 1

In the present example, B6 mice were immunized with INF-01P vaccine plus either Alum, Ribi, or Montanide, or the commercial vaccine (2004-2005 season). Sera was obtained from vaccinated mice one day prior to challenge with virus. Mice were challenged with pathogenic A/HK/1/68-MA20c virus and followed for three weeks post-challenge.

The INF-01P vaccine is based on 4 human influenza sequence discosite construct formulations as shown in Table 19:

TABLE 19
INF-01P influenza vaccine formulation

| | |
|---|---|
| INF-HA-1-V1 | (SEQ ID NO: 1) |
| YACKRGGKSSGSSYPVLNVSY | (SEQ ID NOs: 1 to 16) |
| ----H------------S-TM | |
| INF-HA-1-V2 | (SEQ ID NO: 17) |
| KKGSVHHPSTITEQTSLYVNA | (SEQ ID NOs: 17 to 32) |
| -S-------------T--QQ- | |
| INF-HA-1-V3 | (SEQ ID NO: 33) |
| DVLFSVESPNNKNKDPIDTCD | (SEQ ID NOs: 33 to 48) |
| ------K-V-----ES----- | |
| INF-HA-1-V4 | (SEQ ID NO: 49) |
| YVSVSTSRIASRPKVRGQSGR | (SEQ ID NOs: 49 to 64) |
| --T--S---G---W------- | |

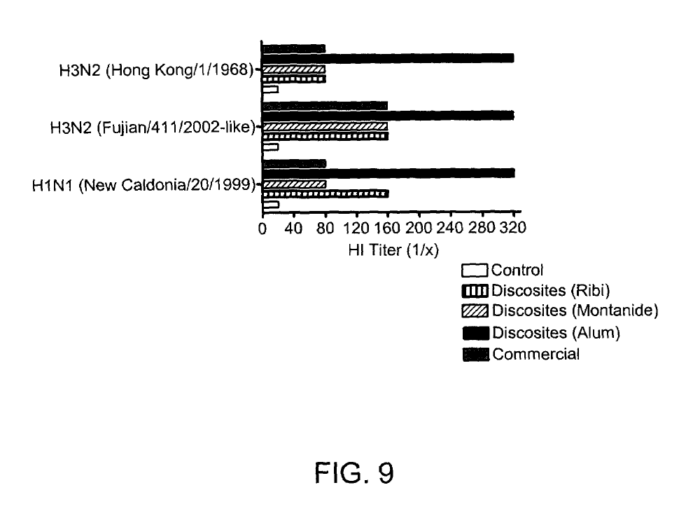

FIG. 9 shows the induction of humoral immunity by INF-01P vaccination as measured by HAI titres. As shown in this example, mice immunized with INF-01P plus Alum vaccine had increased humoral immunity as compared to mice immunized with INF-01P plus Ribi or INF-01P plus Montanide, and compared to the current influenza vaccine.

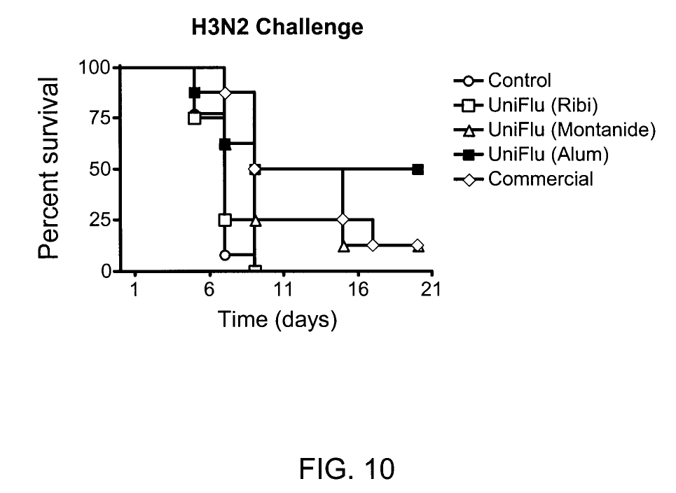

FIG. 10 shows a survival plot of INF-01P-vaccinated mice against challenge with H3N2. In this example, mice immunized with INF-01P plus Alum vaccine are better protected and have a better survival rate against challenge compared to INF-01P plus Ribi or INF-01P plus Montanide.

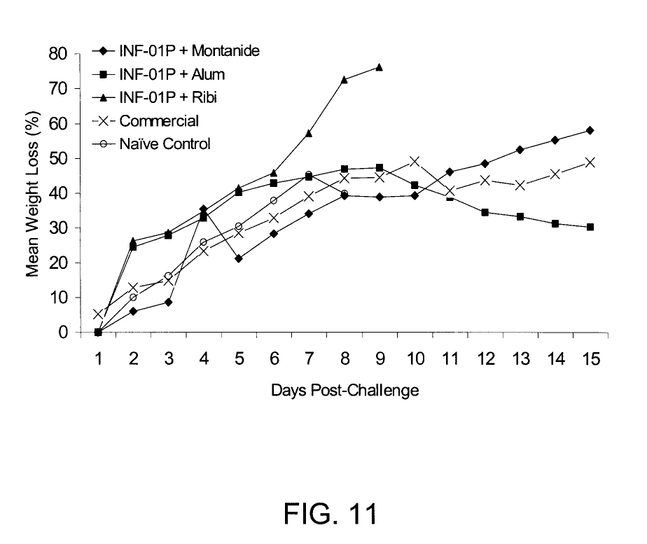

FIG. 11 illustrates percent weight loss in challenged mice vaccinated with INF-01P. As shown in the present example, mice immunized with INF-01P plus Alum were more protected against weight loss than mice immunized with INF-01P plus Ribi or INF-01P plus Montanide.

### Example 2

B6 mice were immunized with INFE-01P (equine flu) vaccine plus either Alum or the commercial vaccine (2004-2005 season). Sera from the mice were tested for HAI activity against several influenza strains (H3N2 A/Hong Kong/1/68g, H1N1 A/FM/1/47, H5N1 A/Hong Kong/213/2003, B/Mass/3/66, and H1N1 A/New Caledonia/20/1999). Sera were obtained after the first vaccination.

The INFE-01P vaccine is based on 4 equine influenza sequence discosite construct formulations as shown in Table 20:

TABLE 20
INFE-01P Equine influenza vaccine formulation

| | |
|---|---|
| INFE-HA-1-V1 | (SEQ ID NO: 185) |
| SACKRRSASSNAAFPQMNKTM | (SEQ ID NOs: 185 to 200) |
| -------------Y---T-SY | |
| INFE-HA-1-V2 | (SEQ ID NO: 201) |
| SSTDNAIHHSSSNQEQTKLYVQE | (SEQ ID NOs: 201 to 216) |
| -N-------P---T-------S- | |
| INFE-HA-1-V3 | (SEQ ID NO: 217) |
| DQFQEESPNNRNFDPDDNCE | (SEQ ID NOs: 217 to 232) |
| ---L-F---T---P------ | |
| INFE-HA-1-V4 | (SEQ ID NO: 233) |
| RITVSTSRPGARPWVRGQSGR | (SEQ ID NOs: 233 to 248) |
| -----S----S--Q-N----- | |

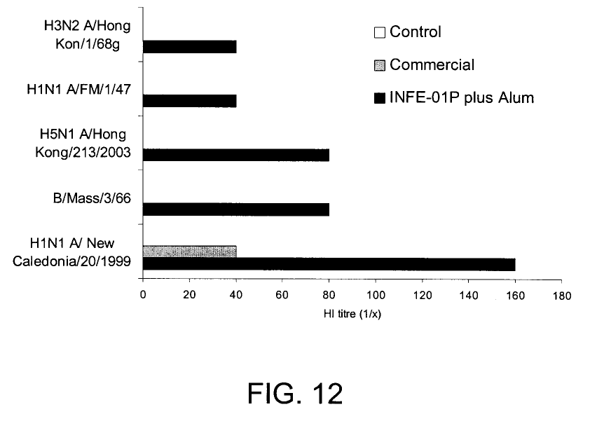

FIG. 12 illustrates humoral immunity in mice immunized with INFE-01P, as measured by HAI titres. As illustrated, humoral immunity was induced in mice immunized with this exemplary equine vaccine formulation against several strains of influenza virus, and as compared to the commercial vaccine or adjuvant only (control) mice.

### Example 3

### Hemagglutination (HAI) Assays

The immunogenicity of the individual and combined discotope constructs was evaluated in mice. Mice immunized with the four discotope constructs collectively developed antibodies that could inhibit viral hemagglutinination activity. Influenza-based discotope constructs were shown to successfully mimic discontinuous epitopes in that antibodies were elicited that inhibited hemagglutination of red blood cells by influenza virus.

A standard HAI assay was used to measure induction of functionally relevant antibodies against HA. Numerous distinct strains of influenza were used to test HAI titres induced by vaccine candidates in order to determine the breadth of immunity induced by the vaccine preparations.

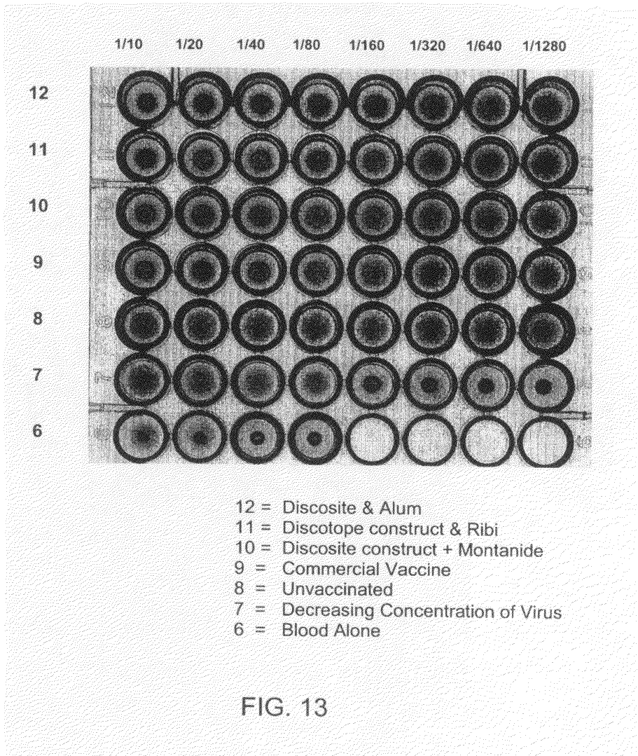

FIG. 13 illustrates the results of a hemagluttination assay performed in murine vaccine study. Each vaccine group received a different vaccine formulation or phosphate buffered saline (negative control). When incubated with virus (H3 Subtype influenza), blood will hemagluttinate (cloudy); when agglutination is protected or inhibited, the RBC remain in a pellet (dark circle).

These results exhibit the strength of hemagluttination inhibition exhibited by the VBI candidate vaccines, as compared to a licensed commercial vaccine for influenza. Note that each of the VBI candidate vaccines and adjuvant combinations exhibit equal or greater hemagluttination inhibition (HI) as does the commercial vaccine (up to 1/40-1/80). The discosite construct immunogens with Alum adjuvant (row 12) demonstrate detectable HI even at dilutions up to 1/320.

TABLE 21
Reciprocal Titre for Different Treatments

| Immunogen | Reciprocal Titre |
|---|---|
| Unvaccinated Control | 10 |
| Commercial Vaccine | 80 |
| Discosite Immunogens + Montanide | 80 |
| Discotope Immunogens + Ribi | 160 |
| Discosite Immunogens + Alum | 320 |

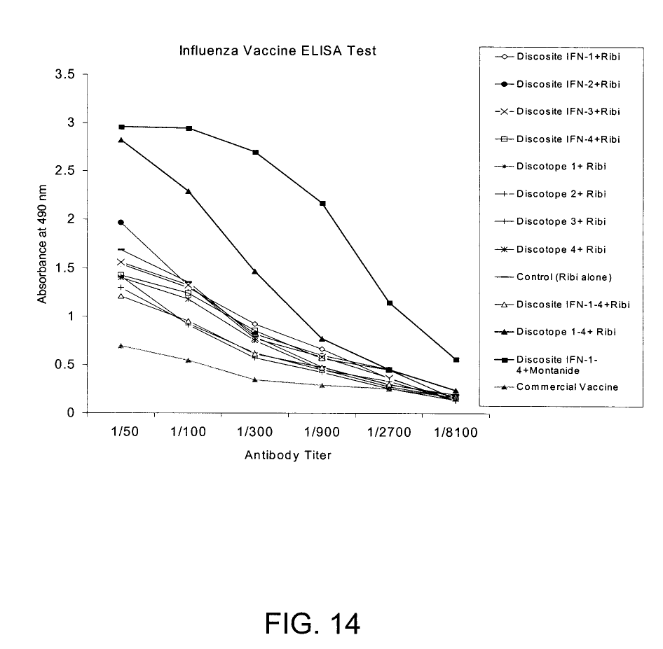

FIG. 14 shows results of the Influenza Vaccine ELISA test. This is based on data presented in Table 22.

TABLE 22
Influenza Vaccine ELISA Test Results

| | 1/50 | 1/100 | 1/300 | 1/900 | 1/2700 | 1/8100 |
|---|---|---|---|---|---|---|
| G1 | 1.538 | 1.295 | 0.921 | 0.665 | 0.358 | 0.134 |
| G2 | 1.965 | 1.319 | 0.818 | 0.595 | 0.455 | 0.145 |
| G3 | 1.559 | 1.321 | 0.771 | 0.583 | 0.367 | 0.121 |
| G4 | 1.427 | 1.24 | 0.854 | 0.564 | 0.446 | 0.149 |
| G5 | 1.258 | 1.13 | 0.667 | 0.456 | 0.335 | 0.141 |
| G6 | 1.298 | 0.926 | 0.623 | 0.45 | 0.329 | 0.13 |
| G7 | 1.416 | 0.912 | 0.571 | 0.421 | 0.251 | 0.135 |
| G8 | 1.4 | 1.177 | 0.753 | 0.453 | 0.271 | 0.16 |
| G9 | 1.686 | 1.355 | 0.795 | 0.477 | 0.291 | 0.193 |
| G10 | 1.21 | 0.95 | 0.613 | 0.475 | 0.296 | 0.176 |
| G11 | 2.82 | 2.29 | 1.467 | 0.77 | 0.455 | 0.236 |
| G12 | 2.959 | 2.944 | 2.699 | 2.174 | 1.146 | 0.559 |
| G13 | 0.694 | 0.547 | 0.343 | 0.289 | 0.253 | 0.16 |

G1 = Discosite construct IFN-1 (SEQ ID NOs: 1 to 16) + Ribi; G2 = Discosite construct IFN-2 (SEQ ID NOs: 17 to 32) + Ribi; G3 = Discosite construct IFN-3 (SEQ ID NOs: 33 to 48) + Ribi; G4 = Discosite construct IFN-4 (SEQ ID NOs: 49 to 64) + Ribi; G5 = Discotope construct 1 (SEQ ID NO: 129) + Ribi; G6 = Discotope construct 2 (SEQ ID NO: 130) + Ribi; G7 = Discotope construct 3 (SEQ ID NO: 131) + Ribi; G8 = Discotope construct 4 (SEQ ID NO: 132) + Ribi; G9 = Control (Ribi alone); G10 = Discosite construct IFN-1-4 + Ribi; G11 = Discotope construct 1-4 + Ribi; G12 = Discosite construct IFN-1-4 + Montanide; and G13 = Commercial Vaccine.

Collectively, these data suggest a fundamentally new approach to influenza vaccine development that results in faster vaccine production and broader protection than current influenza vaccines. This approach may be of value to the development of a pandemic influenza vaccine.

The above-described embodiments of the present invention are intended to be examples only. Alterations, modifications and variations may be effected to the particular embodiments by those of skill in the art without departing from the scope of the invention, which is defined solely by the claims appended hereto.

All references are herein incorporated by reference.

What is claimed is:

1. A peptide-based anti-influenza formulation comprising at least one peptide selected from the group consisting of SEQ ID NOs: 1 to 248.

2. The formulation of claim 1 comprising at least four peptide sequences selected from the group consisting of SEQ ID NOs: 1 to 248.

3. The formulation of claim 1 comprising at least two peptides selected from the group consisting of SEQ ID NOs: 1 to 64 and at least two peptides selected from the group consisting of SEQ ID NOs: 133 to 180.

4. The formulation of claim 3 comprising SEQ ID NOs: 1 to 64 and SEQ ID NOs: 133 to 180.

5. The formulation of claim 1 comprising at least one peptide sequence selected from the group consisting of SEQ ID NOs: 185 to 248.

6. The formulation of claim 1 comprising at least one peptide sequence selected from the group consisting of SEQ ID NOs: 65 to 128.

7. The formulation of claim 1 comprising 2ⁿ peptide sequences from each of at least two of the following groups:
a) SEQ ID NOs: 1 to 16;
b) SEQ ID NOs: 17 to 32;
c) SEQ ID NOs: 33 to 48: or
d) SEQ ID NOs: 49 to 64,
wherein n is 1 to 4.

8. The formulation of claim 1 comprising from at least two of groups a) to d):
a) 2^{m} peptide sequences from SEQ ID NOs: 133 to 140;
b) 2ⁿ peptide sequences from SEQ ID NOs: 141 to 156;
c)2ⁿ peptide sequences from SEQ ID NOs: 157 to 172; or
d) 2^{m} peptide sequences from SEQ ID NOs: 173 to 180,
wherein m is 1 to 3 and n is 1 to 4.

9. The formulation of claim 1 comprising 2ⁿ peptide sequences from each of at least two of the following groups:
a) SEQ ID NOs: 185 to 200;
b) SEQ ID NOs: 201 to 216;
c) SEQ ID NOs: 217 to 232; or
d) SEQ ID NOs: 233 to 248,
wherein n is from 1 to 4.

10. The formulation of claim 1 comprising 2ⁿ peptide sequences from each of at least two of the following groups:
a) SEQ ID NOs: 65 to 80;
b) SEQ ID NOs: 81 to 96;
c) SEQ ID NOs: 97 to 112; or
d) SEQ ID NOs: 113 to 128,
wherein n is from 1 to 4.

11. The formulation of claim 1 comprising at least one of SEQ ID NOs: 129 to 132 or SEQ ID NOs: 181 to 184.

12. The formulation of claim 11 comprising SEQ ID NOs: 129 to 132.

13. The formulation of claim 11 comprising SEQ ID NOs: 181 to 184.

14. The formulation of claim 1 comprising SEQ ID NOs: 1 to 64.

15. The formulation of claim 1 comprising SEQ ID NOs: 185 to 248.

16. A vaccine comprising the formulation of claim 1 together with a pharmaceutically-acceptable diluent or carrier.

17. The vaccine of claim 16 further comprising an adjuvant.

18. The vaccine of claim 17 wherein the adjuvant is alum.

19. Use of the formulation of claim 1 for the preparation of a vaccine for preventing or treating influenza in an animal in need thereof.

20. The use according to claim 19 wherein the animal is human, murine, equine or avian.

21. Use of the formulation of claim 3 for the preparation of a vaccine for treating human influenza.

22. Use of the formulation of claim 5 for the preparation of a vaccine for treating equine influenza.

23. Use of the formulation of claim 6 for the preparation of a vaccine for treating avian influenza.

24. Use of the vaccine of claim 16 for preventing or treating influenza.

25. A vaccine comprising the formulation of claim 3.

26. The vaccine of claim 25 further comprising an adjuvant.

27. The vaccine of claim 26 wherein the adjuvant is alum.

28. A method of preventing or treating influenza in an animal in need thereof comprising administering to the animal an effective amount of the vaccine of claim 16.


SEQUENCE LISTING (248 sequences)

SEQ ID NO: 1 - protein, 21 aa, Haemophilus influenzae
YACKRGGKSSGSSYPVLNVSY

SEQ ID NO: 2 - protein, 21 aa, Haemophilus influenzae
YACKRGGKSSGSSYPVLNVSM

SEQ ID NO: 3 - protein, 21 aa, Haemophilus influenzae
YACKRGGKSSGSSYPVLNVTY

SEQ ID NO: 4 - protein, 21 aa, Haemophilus influenzae
YACKRGGKSSGSSYPVLNVTM

SEQ ID NO: 5 - protein, 21 aa, Haemophilus influenzae
YACKRGGKSSGSSYPVLSVSY

SEQ ID NO: 6 - protein, 21 aa, Haemophilus influenzae
YACKRGGKSSGSSYPVLSVSM

SEQ ID NO: 7 - protein, 21 aa, Haemophilus influenzae
YACKRGGKSSGSSYPVLSVTY

SEQ ID NO: 8 - protein, 21 aa, Haemophilus influenzae
YACKRGGKSSGSSYPVLSVTM

SEQ ID NO: 9 - protein, 21 aa, Haemophilus influenzae
YACKHGGKSSGSSYPVLNVSY

SEQ ID NO: 10 - protein, 21 aa, Haemophilus influenzae
YACKHGGKSSGSSYPVLNVSM

SEQ ID NO: 11 - protein, 21 aa, Haemophilus influenzae
YACKHGGKSSGSSYPVLNVTY

SEQ ID NO: 12 - protein, 21 aa, Haemophilus influenzae
YACKHGGKSSGSSYPVLNVTM

SEQ ID NO: 13 - protein, 21 aa, Haemophilus influenzae
YACKHGGKSSGSSYPVLSVSY

SEQ ID NO: 14 - protein, 21 aa, Haemophilus influenzae
YACKHGGKSSGSSYPVLSVSM

SEQ ID NO: 15 - protein, 21 aa, Haemophilus influenzae
YACKHGGKSSGSSYPVLSVTY

SEQ ID NO: 16 - protein, 21 aa, Haemophilus influenzae
YACKHGGKSSGSSYPVLSVTM

SEQ ID NO: 17 - protein, 21 aa, Haemophilus influenzae
KKGSVHHPSTITEQTSLYVNA

SEQ ID NO: 18 - protein, 21 aa, Haemophilus influenzae
KKGSVHHPSTITEQTSLYVQA

SEQ ID NO: 19 - protein, 21 aa, Haemophilus influenzae
KKGSVHHPSTITEQTSLYQNA

SEQ ID NO: 20 - protein, 21 aa, Haemophilus influenzae
KKGSVHHPSTITEQTSLYQQA

SEQ ID NO: 21 - protein, 21 aa, Haemophilus influenzae
KKGSVHHPSTITEQTTLYVNA

SEQ ID NO: 22 - protein, 21 aa, Haemophilus influenzae
KKGSVHHPSTITEQTTLYVQA

SEQ ID NO: 23 - protein, 21 aa, Haemophilus influenzae
KKGSVHHPSTITEQTTLYQNA

SEQ ID NO: 24 - protein, 21 aa, Haemophilus influenzae
KKGSVHHPSTITEQTTLYQQA

SEQ ID NO: 25 - protein, 21 aa, Haemophilus influenzae
KSGSVHHPSTITEQTSLYVNA

SEQ ID NO: 26 - protein, 21 aa, Haemophilus influenzae
KSGSVHHPSTITEQTSLYVQA

SEQ ID NO: 27 - protein, 21 aa, Haemophilus influenzae
KSGSVHHPSTITEQTSLYQNA

SEQ ID NO: 28 - protein, 21 aa, Haemophilus influenzae
KSGSVHHPSTITEQTSLYQQA

SEQ ID NO: 29 - protein, 21 aa, Haemophilus influenzae
KSGSVHHPSTITEQTTLYVNA

SEQ ID NO: 30 - protein, 21 aa, Haemophilus influenzae
KSGSVHHPSTITEQTTLYVQA

SEQ ID NO: 31 - protein, 21 aa, Haemophilus influenzae
KSGSVHHPSTITEQTTLYQNA

SEQ ID NO: 32 - protein, 21 aa, Haemophilus influenzae
KSGSVHHPSTITEQTTLYQQA

SEQ ID NO: 33 - protein, 21 aa, Haemophilus influenzae
DVLFSVESPNNKNKDPIDTCD

SEQ ID NO: 34 - protein, 21 aa, Haemophilus influenzae
DVLFSVESPNNKNKDSIDTCD

SEQ ID NO: 35 - protein, 21 aa, Haemophilus influenzae
DVLFSVESPNNKNKEPIDTCD

SEQ ID NO: 36 - protein, 21 aa, Haemophilus influenzae
DVLFSVESPNNKNKESIDTCD

SEQ ID NO: 37 - protein, 21 aa, Haemophilus influenzae
DVLFSVESVNNKNKDPIDTCD

SEQ ID NO: 38 - protein, 21 aa, Haemophilus influenzae
DVLFSVESVNNKNKDSIDTCD

SEQ ID NO: 39 - protein, 21 aa, Haemophilus influenzae
DVLFSVESVNNKNKEPIDTCD

SEQ ID NO: 40 - protein, 21 aa, Haemophilus influenzae
DVLFSVESVNNKNKESIDTCD

SEQ ID NO: 41 - protein, 21 aa, Haemophilus influenzae
DVLFSVKSPNNKNKDPIDTCD

SEQ ID NO: 42 - protein, 21 aa, Haemophilus influenzae
DVLFSVKSPNNKNKDSIDTCD

SEQ ID NO: 43 - protein, 21 aa, Haemophilus influenzae
DVLFSVKSPNNKNKEPIDTCD

SEQ ID NO: 44 - protein, 21 aa, Haemophilus influenzae
DVLFSVKSPNNKNKESIDTCD

SEQ ID NO: 45 - protein, 21 aa, Haemophilus influenzae
DVLFSVKSVNNKNKDPIDTCD

SEQ ID NO: 46 - protein, 21 aa, Haemophilus influenzae
DVLFSVKSVNNKNKDSIDTCD

SEQ ID NO: 47 - protein, 21 aa, Haemophilus influenzae
DVLFSVKSVNNKNKEPIDTCD

SEQ ID NO: 48 - protein, 21 aa, Haemophilus influenzae
DVLFSVKSVNNKNKESIDTCD

SEQ ID NO: 49 - protein, 21 aa, Haemophilus influenzae
YVSVSTSRIASRPKVRGQSGR

SEQ ID NO: 50 - protein, 21 aa, Haemophilus influenzae
YVSVSTSRIASRPWVRGQSGR

SEQ ID NO: 51 - protein, 21 aa, Haemophilus influenzae
YVSVSTSRIGSRPKVRGQSGR

SEQ ID NO: 52 - protein, 21 aa, Haemophilus influenzae
YVSVSTSRIGSRPWVRGQSGR

SEQ ID NO: 53 - protein, 21 aa, Haemophilus influenzae
YVSVSSSRIASRPKVRGQSGR

SEQ ID NO: 54 - protein, 21 aa, Haemophilus influenzae
YVSVSSSRIASRPWVRGQSGR

SEQ ID NO: 55 - protein, 21 aa, Haemophilus influenzae
YVSVSSSRIGSRPKVRGQSGR

SEQ ID NO: 56 - protein, 21 aa, Haemophilus influenzae
YVSVSSSRIGSRPWVRGQSGR

SEQ ID NO: 57 - protein, 21 aa, Haemophilus influenzae
YVTVSTSRIASRPKVRGQSGR

SEQ ID NO: 58 - protein, 21 aa, Haemophilus influenzae
YVTVSTSRIASRPWVRGQSGR

SEQ ID NO: 59 - protein, 21 aa, Haemophilus influenzae
YVTVSTSRIGSRPKVRGQSGR

SEQ ID NO: 60 - protein, 21 aa, Haemophilus influenzae
YVTVSTSRIGSRPWVRGQSGR

SEQ ID NO: 61 - protein, 21 aa, Haemophilus influenzae
YVTVSSSRIASRPKVRGQSGR

SEQ ID NO: 62 - protein, 21 aa, Haemophilus influenzae
YVTVSSSRIASRPWVRGQSGR

SEQ ID NO: 63 - protein, 21 aa, Haemophilus influenzae
YVTVSSSRIGSRPKVRGQSGR

SEQ ID NO: 64 - protein, 21 aa, Haemophilus influenzae
YVTVSSSRIGSRPWVRGQSGR

SEQ ID NO: 65 - protein, 21 aa, Haemophilus influenzae
YACKRGGKSSGSSYPVLKVSY

SEQ ID NO: 66 - protein, 21 aa, Haemophilus influenzae
YACKRGGKSSGSSYPVLKVTY

SEQ ID NO: 67 - protein, 21 aa, Haemophilus influenzae
YACKRGGKSSGSSYPVLKRSY

SEQ ID NO: 68 - protein, 21 aa, Haemophilus influenzae
YACKRGGKSSGSSYPVLKRTY

SEQ ID NO: 69 - protein, 21 aa, Haemophilus influenzae
YACKRGGKSSGSSYPVLSVSY

SEQ ID NO: 70 - protein, 21 aa, Haemophilus influenzae
YACKRGGKSSGSSYPVLSVTY

SEQ ID NO: 71 - protein, 21 aa, Haemophilus influenzae
YACKRGGKSSGSSYPVLSRSY

SEQ ID NO: 72 - protein, 21 aa, Haemophilus influenzae
YACKRGGKSSGSSYPVLSRTY

SEQ ID NO: 73 - protein, 21 aa, Haemophilus influenzae
YACKYGGKSSGSSYPVLKVSY

SEQ ID NO: 74 - protein, 21 aa, Haemophilus influenzae
YACKYGGKSSGSSYPVLKVTY

SEQ ID NO: 75 - protein, 21 aa, Haemophilus influenzae
YACKYGGKSSGSSYPVLKRSY

SEQ ID NO: 76 - protein, 21 aa, Haemophilus influenzae
YACKYGGKSSGSSYPVLKRTY

SEQ ID NO: 77 - protein, 21 aa, Haemophilus influenzae
YACKYGGKSSGSSYPVLSVSY

SEQ ID NO: 78 - protein, 21 aa, Haemophilus influenzae
YACKYGGKSSGSSYPVLSVTY

SEQ ID NO: 79 - protein, 21 aa, Haemophilus influenzae
YACKYGGKSSGSSYPVLSRSY

SEQ ID NO: 80 - protein, 21 aa, Haemophilus influenzae
YACKYGGKSSGSSYPVLSRTY

SEQ ID NO: 81 - protein, 21 aa, Haemophilus influenzae
KKGSVHHPSTITEQTSLYVNA

SEQ ID NO: 82 - protein, 21 aa, Haemophilus influenzae
KKGSVHHPSTITEQTSLYVQA

SEQ ID NO: 83 - protein, 21 aa, Haemophilus influenzae
KKGSVHHPSTITEQTSLYQNA

SEQ ID NO: 84 - protein, 21 aa, Haemophilus influenzae
KKGSVHHPSTITEQTSLYQQA

SEQ ID NO: 85 - protein, 21 aa, Haemophilus influenzae
KKGSVHHPSTITEQTKLYVNA

SEQ ID NO: 86 - protein, 21 aa, Haemophilus influenzae
KKGSVHHPSTITEQTKLYVQA

SEQ ID NO: 87 - protein, 21 aa, Haemophilus influenzae
KKGSVHHPSTITEQTKLYQNA

SEQ ID NO: 88 - protein, 21 aa, Haemophilus influenzae
KKGSVHHPSTITEQTKLYQQA

SEQ ID NO: 89 - protein, 21 aa, Haemophilus influenzae
KSGSVHHPSTITEQTSLYVNA

SEQ ID NO: 90 - protein, 21 aa, Haemophilus influenzae
KSGSVHHPSTITEQTSLYVQA

SEQ ID NO: 91 - protein, 21 aa, Haemophilus influenzae
KSGSVHHPSTITEQTSLYQNA

SEQ ID NO: 92 - protein, 21 aa, Haemophilus influenzae
KSGSVHHPSTITEQTSLYQQA

SEQ ID NO: 93 - protein, 21 aa, Haemophilus influenzae
KSGSVHHPSTITEQTKLYVNA

SEQ ID NO: 94 - protein, 21 aa, Haemophilus influenzae
KSGSVHHPSTITEQTKLYVQA

SEQ ID NO: 95 - protein, 21 aa, Haemophilus influenzae
KSGSVHHPSTITEQTKLYQNA

SEQ ID NO: 96 - protein, 21 aa, Haemophilus influenzae
KSGSVHHPSTITEQTKLYQQA

SEQ ID NO: 97 - protein, 21 aa, Haemophilus influenzae
DVLFSVESPNNKNKDPIDTCD

SEQ ID NO: 98 - protein, 21 aa, Haemophilus influenzae
DVLFSVESPNNKNKDEIDTCD

SEQ ID NO: 99 - protein, 21 aa, Haemophilus influenzae
DVLFSVESPNNKNKEPIDTCD

SEQ ID NO: 100 - protein, 21 aa, Haemophilus influenzae
DVLFSVESPNNKNKEEIDTCD

SEQ ID NO: 101 - protein, 21 aa, Haemophilus influenzae
DVLFSVESNNNKNKDPIDTCD

SEQ ID NO: 102 - protein, 21 aa, Haemophilus influenzae
DVLFSVESNNNKNKDEIDTCD

SEQ ID NO: 103 - protein, 21 aa, Haemophilus influenzae
DVLFSVESNNNKNKEPIDTCD

SEQ ID NO: 104 - protein, 21 aa, Haemophilus influenzae
DVLFSVESNNNKNKEEIDTCD

SEQ ID NO: 105 - protein, 21 aa, Haemophilus influenzae
DVLFSVPSPNNKNKDPIDTCD

SEQ ID NO: 106 - protein, 21 aa, Haemophilus influenzae
DVLFSVPSPNNKNKDEIDTCD

SEQ ID NO: 107 - protein, 21 aa, Haemophilus influenzae
DVLFSVPSPNNKNKEPIDTCD

SEQ ID NO: 108 - protein, 21 aa, Haemophilus influenzae
DVLFSVPSPNNKNKEEIDTCD

SEQ ID NO: 109 - protein, 21 aa, Haemophilus influenzae
DVLFSVPSNNNKNKDPIDTCD

SEQ ID NO: 110 - protein, 21 aa, Haemophilus influenzae
DVLFSVPSNNNKNKDEIDTCD

SEQ ID NO: 111 - protein, 21 aa, Haemophilus influenzae
DVLFSVPSNNNKNKEPIDTCD

SEQ ID NO: 112 - protein, 21 aa, Haemophilus influenzae
DVLFSVPSNNNKNKEEIDTCD

SEQ ID NO: 113 - protein, 21 aa, Haemophilus influenzae
YVSVSTSRIASRPKVRGQSGR

SEQ ID NO: 114 - protein, 21 aa, Haemophilus influenzae
YVSVSTSRIASRPWVRGQSGR

SEQ ID NO: 115 - protein, 21 aa, Haemophilus influenzae
YVSVSTSRIGSRPKVRGQSGR

SEQ ID NO: 116 - protein, 21 aa, Haemophilus influenzae
YVSVSTSRIGSRPWVRGQSGR

SEQ ID NO: 117 - protein, 21 aa, Haemophilus influenzae
YVSVSSSRIASRPKVRGQSGR

SEQ ID NO: 118 - protein, 21 aa, Haemophilus influenzae
YVSVSSSRIASRPWVRGQSGR

SEQ ID NO: 119 - protein, 21 aa, Haemophilus influenzae
YVSVSSSRIGSRPKVRGQSGR

SEQ ID NO: 120 - protein, 21 aa, Haemophilus influenzae
YVSVSSSRIGSRPWVRGQSGR

SEQ ID NO: 121 - protein, 21 aa, Haemophilus influenzae
YVTVSTSRIASRPKVRGQSGR

SEQ ID NO: 122 - protein, 21 aa, Haemophilus influenzae
YVTVSTSRIASRPWVRGQSGR

SEQ ID NO: 123 - protein, 21 aa, Haemophilus influenzae
YVTVSTSRIGSRPKVRGQSGR

SEQ ID NO: 124 - protein, 21 aa, Haemophilus influenzae
YVTVSTSRIGSRPWVRGQSGR

SEQ ID NO: 125 - protein, 21 aa, Haemophilus influenzae
YVTVSSSRIASRPKVRGQSGR

SEQ ID NO: 126 - protein, 21 aa, Haemophilus influenzae
YVTVSSSRIASRPWVRGQSGR

SEQ ID NO: 127 - protein, 21 aa, Haemophilus influenzae
YVTVSSSRIGSRPKVRGQSGR

SEQ ID NO: 128 - protein, 21 aa, Haemophilus influenzae
YVTVSSSRIGSRPWVRGQSGR

SEQ ID NO: 129 - protein, 21 aa, Haemophilus influenzae
YACKRGGKSSGSSYPVLNVSY

SEQ ID NO: 130 - protein, 21 aa, Haemophilus influenzae
KKGSVHHPSTITEQTSLYVNA

SEQ ID NO: 131 - protein, 21 aa, Haemophilus influenzae
DVLFSVESPNNKNKDPIDTCD

SEQ ID NO: 132 - protein, 21 aa, Haemophilus influenzae
YVSVSTSRIASRPKVRGQSGR

SEQ ID NO: 133 - protein, 27 aa, Haemophilus influenzae
GSCPNATNRNGDNNKTAINPLTVEVPY

SEQ ID NO: 134 - protein, 27 aa, Haemophilus influenzae
GSCPNATNRNGDNNKTATNPLTVEVPY

SEQ ID NO: 135 - protein, 27 aa, Haemophilus influenzae
GSCPNATNRSGDNNKTAINPLTVEVPY

SEQ ID NO: 136 - protein, 27 aa, Haemophilus influenzae
GSCPNATNRSGDNNKTATNPLTVEVPY

SEQ ID NO: 137 - protein, 27 aa, Haemophilus influenzae
GSCPNATSRNGDNNKTAINPLTVEVPY

SEQ ID NO: 138 - protein, 27 aa, Haemophilus influenzae
GSCPNATSRNGDNNKTATNPLTVEVPY

SEQ ID NO: 139 - protein, 27 aa, Haemophilus influenzae
GSCPNATSRSGDNNKTAINPLTVEVPY

SEQ ID NO: 140 - protein, 27 aa, Haemophilus influenzae
GSCPNATSRSGDNNKTATNPLTVEVPY

SEQ ID NO: 141 - protein, 21 aa, Haemophilus influenzae
PKDNFHSDNKTQMERLYGDSN

SEQ ID NO: 142 - protein, 21 aa, Haemophilus influenzae
PKDNFHSDNKTQMENLYGDSN

SEQ ID NO: 143 - protein, 21 aa, Haemophilus influenzae
PKDNFHSDNKTQMKRLYGDSN

SEQ ID NO: 144 - protein, 21 aa, Haemophilus influenzae
PKDNFHSDNKTQMKNLYGDSN

SEQ ID NO: 145 - protein, 21 aa, Haemophilus influenzae
PNDNFHSDNKTQMERLYGDSN

SEQ ID NO: 146 - protein, 21 aa, Haemophilus influenzae
PNDNFHSDNKTQMENLYGDSN

SEQ ID NO: 147 - protein, 21 aa, Haemophilus influenzae
PNDNFHSDNKTQMKRLYGDSN

SEQ ID NO: 148 - protein, 21 aa, Haemophilus influenzae
PNDNFHSDNKTQMKNLYGDSN

SEQ ID NO: 149 - protein, 21 aa, Haemophilus influenzae
RKDNFHSDNKTQMERLYGDSN

SEQ ID NO: 150 - protein, 21 aa, Haemophilus influenzae
RKDNFHSDNKTQMENLYGDSN

SEQ ID NO: 151 - protein, 21 aa, Haemophilus influenzae
RKDNFHSDNKTQMKRLYGDSN

SEQ ID NO: 152 - protein, 21 aa, Haemophilus influenzae
RKDNFHSDNKTQMKNLYGDSN

SEQ ID NO: 153 - protein, 21 aa, Haemophilus influenzae
RNDNFHSDNKTQMERLYGDSN

SEQ ID NO: 154 - protein, 21 aa, Haemophilus influenzae
RNDNFHSDNKTQMENLYGDSN

SEQ ID NO: 155 - protein, 21 aa, Haemophilus influenzae
RNDNFHSDNKTQMKRLYGDSN

SEQ ID NO: 156 - protein, 21 aa, Haemophilus influenzae
RNDNFHSDNKTQMKNLYGDSN

SEQ ID NO: 157 - protein, 27 aa, Haemophilus influenzae
RGKLCPNCFNCTDIICSEGEDLPLIGE

SEQ ID NO: 158 - protein, 27 aa, Haemophilus influenzae
RGKLCPNCFNCTDIICSKGEDLPLIGE

SEQ ID NO: 159 - protein, 27 aa, Haemophilus influenzae
RGKLCPNCFNCTDIICTEGEDLPLIGE

SEQ ID NO: 160 - protein, 27 aa, Haemophilus influenzae
RGKLCPNCFNCTDIICTKGEDLPLIGE

SEQ ID NO: 161 - protein, 27 aa, Haemophilus influenzae
RGKLCPNCFNCTDLICSEGEDLPLIGE

SEQ ID NO: 162 - protein, 27 aa, Haemophilus influenzae
RGKLCPNCFNCTDLICSKGEDLPLIGE

SEQ ID NO: 163 - protein, 27 aa, Haemophilus influenzae
RGKLCPNCFNCTDLICTEGEDLPLIGE

SEQ ID NO: 164 - protein, 27 aa, Haemophilus influenzae
RGKLCPNCFNCTDLICTKGEDLPLIGE

SEQ ID NO: 165 - protein, 27 aa, Haemophilus influenzae
RGKLCPNCLNCTDIICSEGEDLPLIGE

SEQ ID NO: 166 - protein, 27 aa, Haemophilus influenzae
RGKLCPNCLNCTDIICSKGEDLPLIGE

SEQ ID NO: 167 - protein, 27 aa, Haemophilus influenzae
RGKLCPNCLNCTDIICTEGEDLPLIGE

SEQ ID NO: 168 - protein, 27 aa, Haemophilus influenzae
RGKLCPNCLNCTDIICTKGEDLPLIGE

SEQ ID NO: 169 - protein, 27 aa, Haemophilus influenzae
RGKLCPNCLNCTDLICSEGEDLPLIGE

SEQ ID NO: 170 - protein, 27 aa, Haemophilus influenzae
RGKLCPNCLNCTDLICSKGEDLPLIGE

SEQ ID NO: 171 - protein, 27 aa, Haemophilus influenzae
RGKLCPNCLNCTDLICTEGEDLPLIGE

SEQ ID NO: 172 - protein, 27 aa, Haemophilus influenzae
RGKLCPNCLNCTDLICTKGEDLPLIGE

SEQ ID NO: 173 - protein, 26 aa, Haemophilus influenzae
KFTSSANGIGGFPNQTEDEGLKQSGR

SEQ ID NO: 174 - protein, 26 aa, Haemophilus influenzae
KFTSSANGIGGFPNQTEDEGLPQSGR

SEQ ID NO: 175 - protein, 26 aa, Haemophilus influenzae
KFTSSANGIGGFPNQTEDGGLKQSGR

SEQ ID NO: 176 - protein, 26 aa, Haemophilus influenzae
KFTSSANGIGGFPNQTEDGGLPQSGR

SEQ ID NO: 177 - protein, 26 aa, Haemophilus influenzae
KFTSSANGIGGFPDQTEDEGLKQSGR

SEQ ID NO: 178 - protein, 26 aa, Haemophilus influenzae
KFTSSANGIGGFPDQTEDEGLPQSGR

SEQ ID NO: 179 - protein, 26 aa, Haemophilus influenzae
KFTSSANGIGGFPDQTEDGGLKQSGR

SEQ ID NO: 180 - protein, 26 aa, Haemophilus influenzae
KFTSSANGIGGFPDQTEDGGLPQSGR

SEQ ID NO: 181 - protein, 27 aa, Haemophilus influenzae
GSCPNVANGNGDNNKTAINPVTVEVPY

SEQ ID NO: 182 - protein, 21 aa, Haemophilus influenzae
PKDNFHSDDKTQMERLYGDSN

SEQ ID NO: 183 - protein, 27 aa, Haemophilus influenzae
RGKLCPNCFNCTDIICSEGEDLPLIGE

SEQ ID NO: 184 - protein, 26 aa, Haemophilus influenzae
KFTSSANGIGGFPNQTEDEGLKQSGR

SEQ ID NO: 185 - protein, 21 aa, Haemophilus influenzae
SACKRRSASSNAAFPQMNKTM

SEQ ID NO: 186 - protein, 21 aa, Haemophilus influenzae
SACKRRSASSNAAFPQMNKTY

SEQ ID NO: 187 - protein, 21 aa, Haemophilus influenzae
SACKRRSASSNAAFPQMNKSM

SEQ ID NO: 188 - protein, 21 aa, Haemophilus influenzae
SACKRRSASSNAAFPQMNKSY

SEQ ID NO: 189 - protein, 21 aa, Haemophilus influenzae
SACKRRSASSNAAFPQMTKTM

SEQ ID NO: 190 - protein, 21 aa, Haemophilus influenzae
SACKRRSASSNAAFPQMTKTY

SEQ ID NO: 191 - protein, 21 aa, Haemophilus influenzae
SACKRRSASSNAAFPQMTKSM

SEQ ID NO: 192 - protein, 21 aa, Haemophilus influenzae
SACKRRSASSNAAFPQMTKSY

SEQ ID NO: 193 - protein, 21 aa, Haemophilus influenzae
SACKRRSASSNAAYPQMNKTM

SEQ ID NO: 194 - protein, 21 aa, Haemophilus influenzae
SACKRRSASSNAAYPQMNKTY

SEQ ID NO: 195 - protein, 21 aa, Haemophilus influenzae
SACKRRSASSNAAYPQMNKSM

SEQ ID NO: 196 - protein, 21 aa, Haemophilus influenzae
SACKRRSASSNAAYPQMNKSY

SEQ ID NO: 197 - protein, 21 aa, Haemophilus influenzae
SACKRRSASSNAAYPQMTKTM

SEQ ID NO: 198 - protein, 21 aa, Haemophilus influenzae
SACKRRSASSNAAYPQMTKTY

SEQ ID NO: 199 - protein, 21 aa, Haemophilus influenzae
SACKRRSASSNAAYPQMTKSM

SEQ ID NO: 200 - protein, 21 aa, Haemophilus influenzae
SACKRRSASSNAAYPQMTKSY

SEQ ID NO: 201 - protein, 23 aa, Haemophilus influenzae
SSTDNAIHHSSSNQEQTKLYVQE

SEQ ID NO: 202 - protein, 23 aa, Haemophilus influenzae
SSTDNAIHHSSSNQEQTKLYVSE

SEQ ID NO: 203 - protein, 23 aa, Haemophilus influenzae
SSTDNAIHHSSSNTEQTKLYVQE

SEQ ID NO: 204 - protein, 23 aa, Haemophilus influenzae
SSTDNAIHHSSSNTEQTKLYVSE

SEQ ID NO: 205 - protein, 23 aa, Haemophilus influenzae
SSTDNAIHHPSSNQEQTKLYVQE

SEQ ID NO: 206 - protein, 23 aa, Haemophilus influenzae
SSTDNAIHHPSSNQEQTKLYVSE

SEQ ID NO: 207 - protein, 23 aa, Haemophilus influenzae
SSTDNAIHHPSSNTEQTKLYVQE

SEQ ID NO: 208 - protein, 23 aa, Haemophilus influenzae
SSTDNAIHHPSSNTEQTKLYVSE

SEQ ID NO: 209 - protein, 23 aa, Haemophilus influenzae
SNTDNAIHHSSSNQEQTKLYVQE

SEQ ID NO: 210 - protein, 23 aa, Haemophilus influenzae
SNTDNAIHHSSSNQEQTKLYVSE

SEQ ID NO: 211 - protein, 23 aa, Haemophilus influenzae
SNTDNAIHHSSSNTEQTKLYVQE

SEQ ID NO: 212 - protein, 23 aa, Haemophilus influenzae
SNTDNAIHHSSSNTEQTKLYVSE

SEQ ID NO: 213 - protein, 23 aa, Haemophilus influenzae
SNTDNAIHHPSSNQEQTKLYVQE

SEQ ID NO: 214 - protein, 23 aa, Haemophilus influenzae
SNTDNAIHHPSSNQEQTKLYVSE

SEQ ID NO: 215 - protein, 23 aa, Haemophilus influenzae
SNTDNAIHHPSSNTEQTKLYVQE

SEQ ID NO: 216 - protein, 23 aa, Haemophilus influenzae
SNTDNAIHHPSSNTEQTKLYVSE

SEQ ID NO: 217 - protein, 20 aa, Haemophilus influenzae
DQFQEESPNNRNFDPDDNCE

SEQ ID NO: 218 - protein, 20 aa, Haemophilus influenzae
DQFQEESPNNRNFPPDDNCE

SEQ ID NO: 219 - protein, 20 aa, Haemophilus influenzae
DQFQEESPNTRNFDPDDNCE

SEQ ID NO: 220 - protein, 20 aa, Haemophilus influenzae
DQFQEESPNTRNFPPDDNCE

SEQ ID NO: 221 - protein, 20 aa, Haemophilus influenzae
DQFQEFSPNNRNFDPDDNCE

SEQ ID NO: 222 - protein, 20 aa, Haemophilus influenzae
DQFQEFSPNNRNFPPDDNCE

SEQ ID NO: 223 - protein, 20 aa, Haemophilus influenzae
DQFQEFSPNTRNFDPDDNCE

SEQ ID NO: 224 - protein, 20 aa, Haemophilus influenzae
DQFQEFSPNTRNFPPDDNCE

SEQ ID NO: 225 - protein, 20 aa, Haemophilus influenzae
DQFLEESPNNRNFDPDDNCE

SEQ ID NO: 226 - protein, 20 aa, Haemophilus influenzae
DQFLEESPNNRNFPPDDNCE

SEQ ID NO: 227 - protein, 20 aa, Haemophilus influenzae
DQFLEESPNTRNFDPDDNCE

SEQ ID NO: 228 - protein, 20 aa, Haemophilus influenzae
DQFLEESPNTRNFPPDDNCE

SEQ ID NO: 229 - protein, 20 aa, Haemophilus influenzae
DQFLEFSPNNRNFDPDDNCE

SEQ ID NO: 230 - protein, 20 aa, Haemophilus influenzae
DQFLEFSPNNRNFPPDDNCE

SEQ ID NO: 231 - protein, 20 aa, Haemophilus influenzae
DQFLEFSPNTRNFDPDDNCE

SEQ ID NO: 232 - protein, 20 aa, Haemophilus influenzae
DQFLEFSPNTRNFPPDDNCE

SEQ ID NO: 233 - protein, 21 aa, Haemophilus influenzae
RITVSTSRPGARPWVRGQSGR

SEQ ID NO: 234 - protein, 21 aa, Haemophilus influenzae
RITVSTSRPGARPWVNGQSGR

SEQ ID NO: 235 - protein, 21 aa, Haemophilus influenzae
RITVSTSRPGARPQVRGQSGR

SEQ ID NO: 236 - protein, 21 aa, Haemophilus influenzae
RITVSTSRPGARPQVNGQSGR

SEQ ID NO: 237 - protein, 21 aa, Haemophilus influenzae
RITVSTSRPGSRPWVRGQSGR

SEQ ID NO: 238 - protein, 21 aa, Haemophilus influenzae
RITVSTSRPGSRPWVNGQSGR

SEQ ID NO: 239 - protein, 21 aa, Haemophilus influenzae
RITVSTSRPGSRPQVRGQSGR

SEQ ID NO: 240 - protein, 21 aa, Haemophilus influenzae
RITVSTSRPGSRPQVNGQSGR

SEQ ID NO: 241 - protein, 21 aa, Haemophilus influenzae
RITVSSSRPGARPWVRGQSGR

SEQ ID NO: 242 - protein, 21 aa, Haemophilus influenzae
RITVSSSRPGARPWVNGQSGR

SEQ ID NO: 243 - protein, 21 aa, Haemophilus influenzae
RITVSSSRPGARPQVRGQSGR

SEQ ID NO: 244 - protein, 21 aa, Haemophilus influenzae
RITVSSSRPGARPQVNGQSGR

SEQ ID NO: 245 - protein, 21 aa, Haemophilus influenzae
RITVSSSRPGSRPWVRGQSGR

SEQ ID NO: 246 - protein, 21 aa, Haemophilus influenzae
RITVSSSRPGSRPWVNGQSGR

SEQ ID NO: 247 - protein, 21 aa, Haemophilus influenzae
RITVSSSRPGSRPQVRGQSGR

SEQ ID NO: 248 - protein, 21 aa, Haemophilus influenzae
RITVSSSRPGSRPQVNGQSGR